(19) United States
(12) Patent Application Publication
(10) Pub. No.: US 2005/0143430 A1
(43) Pub. Date: Jun. 30, 2005

(54) Catechol bioisosteres

(76) Inventors: Aviv Gazit, Jerusalem (IL); Alexander Levitzki, Jerusalem (IL); Galia Blum, Jerusalem (IL)

Correspondence Address: WINSTON & STRAWN LLP, 1700 K STREET, N.W., WASHINGTON, DC, 20006, US

(21) Appl. No.: 10/914,010

(22) Filed: Aug. 5, 2004

Related U.S. Application Data
(63) Continuation of application No. PCT/IL03/00094, filed on Feb. 5, 2003.
(60) Provisional application No. 60/354,153, filed on Feb. 6, 2002.

(51) Int. Cl. C07D 263/58; C07D 235/24; A61K 31/424; A61K 31/4184

(52) U.S. Cl. 514/375; 514/384; 548/221; 548/306.4

(57) ABSTRACT

The present invention provides catechol bioisostere compounds which are potent inhibitors of protein tyrosine kinases (PTKs). The present invention further provides methods of inhibiting PTKs, for example receptor protein tyrosine kinases (RTKs), comprising administering the catechol bioisosteres. The catechol bioisostere compounds are useful in treating or preventing PTK-related disease states, particularly protein tyrosine kinase related disorders which are associated with defects in signaling pathways mediated by PTKs.

### CROSS-REFERENCE TO RELATED APPLICATIONS

This application is a continuation of International application PCT/IL03/00094, filed Feb. 5, 2003, which claims the benefit of U.S. application Ser. No. 60/354,153, filed Feb. 6, 2002. The entire content of the aforementioned applications is expressly incorporated herein by reference thereto.

### FIELD OF THE INVENTION

The present invention is directed to catechol bioisosteres, their preparation, pharmaceutical compositions containing these compounds, and their use in the treatment of protein tyrosine kinase related disorders.

### BACKGROUND OF THE INVENTION

Protein tyrosine kinases (PTKs) are a family of enzymes, which transfer the γ-phosphate of ATP to the side chain of tyrosine residues on substrate proteins. PTKs are involved in a variety of key cellular processes, including signal transduction and growth regulation. The phosphorylation of substrates by PTKs are key events in cellular signaling.

One class of PTKs are the receptor tyrosine kinases (RTKs). These kinases belong to a family of transmembrane proteins and have been implicated in cellular signaling pathways. The predominant biological activity of some receptor tyrosine kinases is the stimulation of cell growth and proliferation, while other receptor tyrosine kinases are involved in arresting growth and promoting differentiation. In some instances, a single tyrosine kinase can inhibit, or stimulate, cell proliferation depending on the cellular environment in which it is expressed. (Schlessinger, J. and Ullrich, A., Neuron (1992) 9(3): 383-391, 1992.) RTKs include the receptors for platelet-derived growth factor (PDGF), fibroblast growth factor (FGF), hepatocyte growth factor (HGF), insulin, insulin-like growth factor-1 (IGF-1), nerve growth factor (NGF), vascular endothelial growth factor (VEGF), and macrophage colony stimulating factor (M-CSF).

Receptor tyrosine kinases are composed of at least three domains: an extracellular glycosylated ligand binding domain, a transmembrane domain and a cytoplasmic catalytic domain that can phosphorylate tyrosine residues. Ligand binding to membrane-bound receptors induces the formation of receptor dimers and allosteric changes that activate the intracellular kinase domains and result in the self-phosphorylation (autophosphorylation and/or transphosphorylation) of the receptor on tyrosine residues. Receptor phosphorylation stimulates a physical association of the activated receptor with target molecules. Some of the target molecules are, in turn, phosphorylated, which transmits the signal to the cytoplasm. The secondary signal transducer molecules generated by activated receptors result in a signal cascade that regulates cell functions such as cell division or differentiation. Reviews describing intracellular signal transduction include Aaronson, S. A., Science (1991), 254: 1146-1153; Schlessinger, J. Trends Biochem. Sci. (1988) 13: 443-447, 1988; and Ullrich, A., and Schlessinger, J., Cell (1990) 61: 203-212.

Various cell proliferative disorders have been associated with defects in various signaling pathways mediated by PTKs. Enhanced activities of PTKs resulting from overexpression of the normal kinase or due to activating mutations, are a hallmark of many diseases involving cellular proliferation, including cancer. Examples of specific receptor tyrosine kinases associated with cell proliferative disorders include, platelet derived growth factor receptor (PDGFr), epidermal growth factor receptor (EGFr), and the related HER2.

The involvement of PTKs in disease states identifies them as targets for antiproliferative drugs. Indeed, numerous PTK blockers have been described, and their mechanism of action studied (Levitzki, A.; et al. Science (1995), 267, 1782-88; Posner et al. Mol. Pharmacol. (1994), 45, 673-683). Recently Applicants have developed a family of PTK inhibitors, named tyrphostins, designed to mimic the tyrosine substrate (Levitzki et al (1995); Levitzki et al; Biochem. Pharm. (1990), 40, 913-920; Levitzki et al. FASEB J. (1992), 6, 3275-3282; U.S. Pat. Nos. 5,217,999 and 5,773,476). The pharmacophores of these tyrphostins, and specifically the tyrphostins of the benzylidene malononitrile type, are hydrophilic catechol ring and the more lipophilic substituted cyano-vinyl radical. Kinetic studies have shown that some tyrphostin compounds are pure competitive inhibitors vis-à-vis the tyrosine substrates and non-competitive inhibitors vis-à-vis ATP site (Yaish et al. Science (1988) 242, 933-935; Gazit et al. J. Med Chem. (1989), 32, 2344-2352), while many tyrphostins shown competitive inhibition against both the substrate and ATP (Posner et al (1994)).

In a related group of tyrphostins, the hydrophilic catechol ring was exchanged by lipophilic dichloro or dimethoxy phenyl to yield EGFr kinase inhibitors, effective in the low micromolar range (Yoneda et al. Cancer Res. (1991), 51, 4430-4435).

Bioisosteres is a useful concept in drug design. The substitution of certain pharmacophores with chemically different but biologically isoactive pharmacophores results in the preparation of new active entities with modified and improved properties. Tyrphostins of the benzylidene malononitrile chemical class contain the catechol pharmacophore, which is sensitive to oxidation. There is an urgent need to develop new analogs of tyrphostins which are stable towards oxidation, and which are potent inhibitors of protein tyrosine kinases.

### SUMMARY OF THE INVENTION

The present invention provides catechol bioisostere compounds which are potent inhibitors of protein tyrosine kinases (PTKs). These compounds are useful in inhibiting protein tyrosine kinases and are particularly useful in treating protein kinase related disease states as defined herein.

The present invention provides a compound represented by the structure of formula 1: 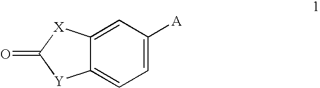
wherein X and Y are independently NR₁ or O, wherein R₁ is H or alkyl; A is a group represented by the formula: 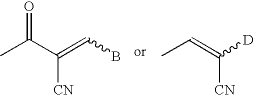
   B is phenyl which is unsubstituted or substituted by one or more OR₂ or COOR₃ wherein R₂ and R₃ are independently hydrogen or alkyl, or B is a group represented by the formula: 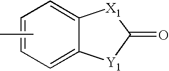
wherein X₁ and Y₁ are independently NR₁ or O, wherein R₁ is H or alkyl; D is CN; C(═O)R₄ wherein R₄ is an alkyl, aralkyl or aryl which is unsubstituted or substituted by one or more OR₅, wherein R₅ is hydrogen or alkyl; or D is C(═O)NR₆R₇ wherein R₆ and R₇ are independently hydrogen or an optionally substituted alkyl, aralkyl or aryl.

The present invention further provides a compound represented by the structure of formula 2.

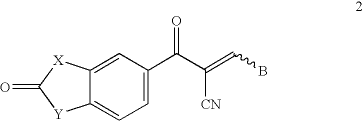

The present invention further provides a compound represented by the structure of formula 3, wherein R₈ R₉ are independently hydroxy, alkoxy or COOH.

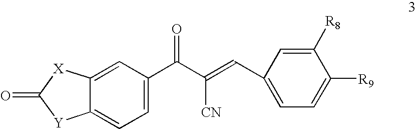

The present invention further provides a compound represented by the structure of formula 4.

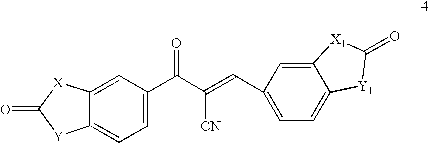

The present invention further provides a compound represented by the structure of formula 5.

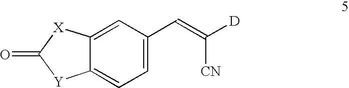

In one embodiment, X and Y are NR₁. In another embodiment, X and Y are NH. In another embodiment, X is NR₁ and Y is NH. In another embodiment, X is NH and Y is NR₁. In another embodiment, X and Y are O. In another embodiment, X is O and Y is NR₁. In another embodiment, X is NR₁ and Y is O. In another embodiment, X is O and Y is NH. In another embodiment, X is NH and Y is O.

In one embodiment, X₁ and Y₁ are NR₁. In another embodiment, X₁ and Y₁ are NH. In another embodiment, X₁ is NR₁ and Y₁ is NH. In another embodiment, X₁ is NH and Y₁ is NR₁. In another embodiment, X₁ and Y₁ are O. In another embodiment, X₁ is O and Y₁ is NR₁. In another embodiment, X₁ is NR₁ and Y₁ is O. In another embodiment, X₁ is O and Y₁ is NH. In another embodiment, X₁ is NH and Y₁ is O.

The present invention further provides a compound selected from the group consisting of:

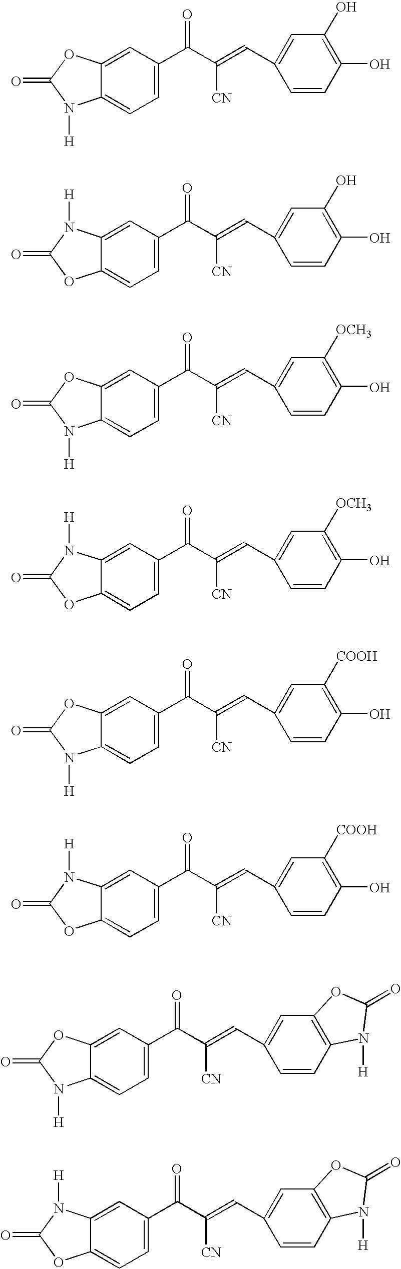

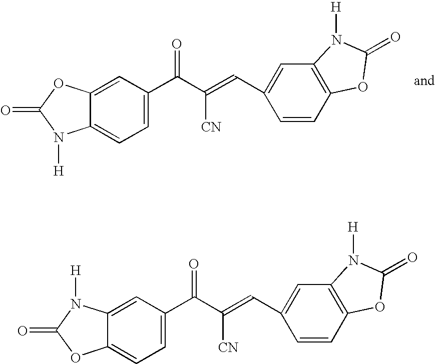

The present invention further provides a compound selected from the group consisting of:

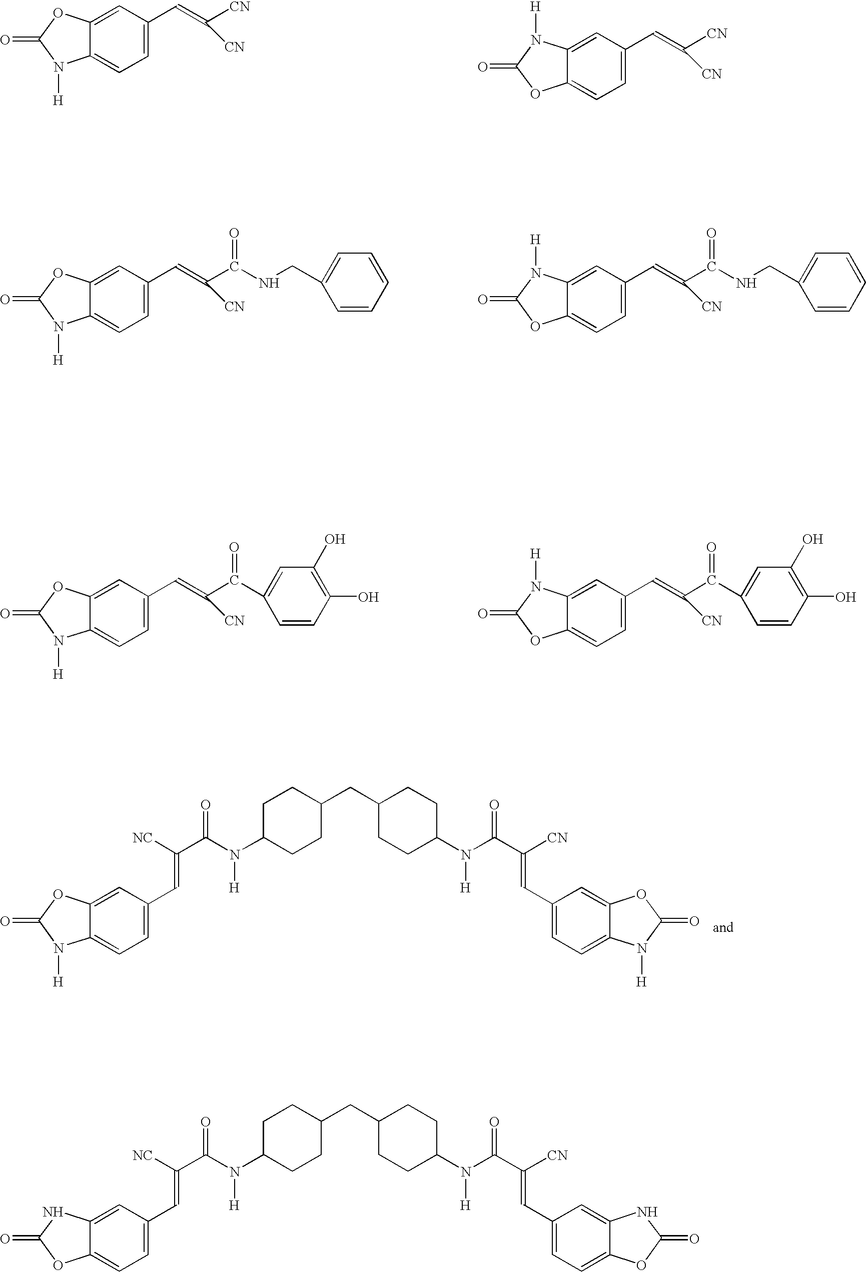

The present invention further provides pharmaceutical compositions comprising any of the compounds represented by formulas 1-5, and a pharmaceutically acceptable carrier or excipient.

The present invention further provides a method of inhibiting a protein tyrosine kinase (PTK) comprising contacting the PTK with an effective inhibitory amount of a compound represented by any of formulas 1-5.

The present invention further provides a method of inhibiting a protein tyrosine kinase (PTK) in a subject comprising the step of administering to the subject a therapeutically effective amount of any of the compounds represented by formulas 1-5. In another embodiment, the method comprises administering to the subject a pharmaceutical composition comprising a therapeutically effective amount of any of the compounds represented by formulas 1-5, and a pharmaceutically acceptable excipient.

The present invention further provides a method of treating or preventing a protein tyrosine kinase (PTK) related disorder in a subject comprising the step of administering to the subject a therapeutically effective amount of any of the compounds represented by formulas 1-5. In another embodiment, the method comprises administering to the subject a pharmaceutical composition comprising a therapeutically effective amount of any of the compounds represented by formulas 1-5; and a pharmaceutically acceptable excipient. In one embodiment, the PTK related disorder is a cell proliferative disorder, a metabolic disorder or a fibrotic disorder. In another embodiment, the PTK related disorder is cancer.

In one embodiment, the compound which is effective at inhibiting protein tyrosine kinase is selected from the group consisting of:

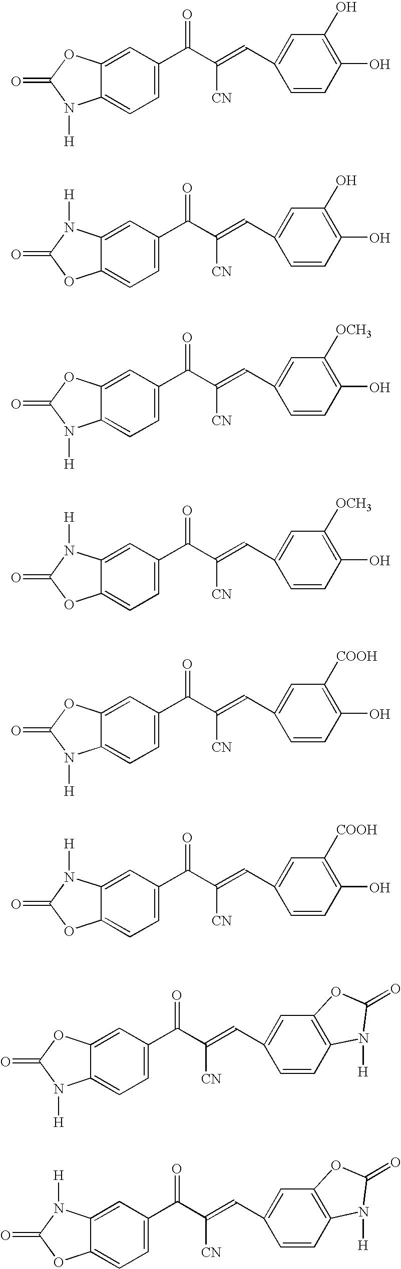

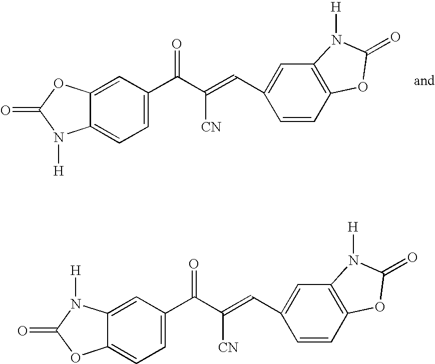

In another embodiment, the compound which is effective at inhibiting protein tyrosine kinase is selected from the group consisting of:

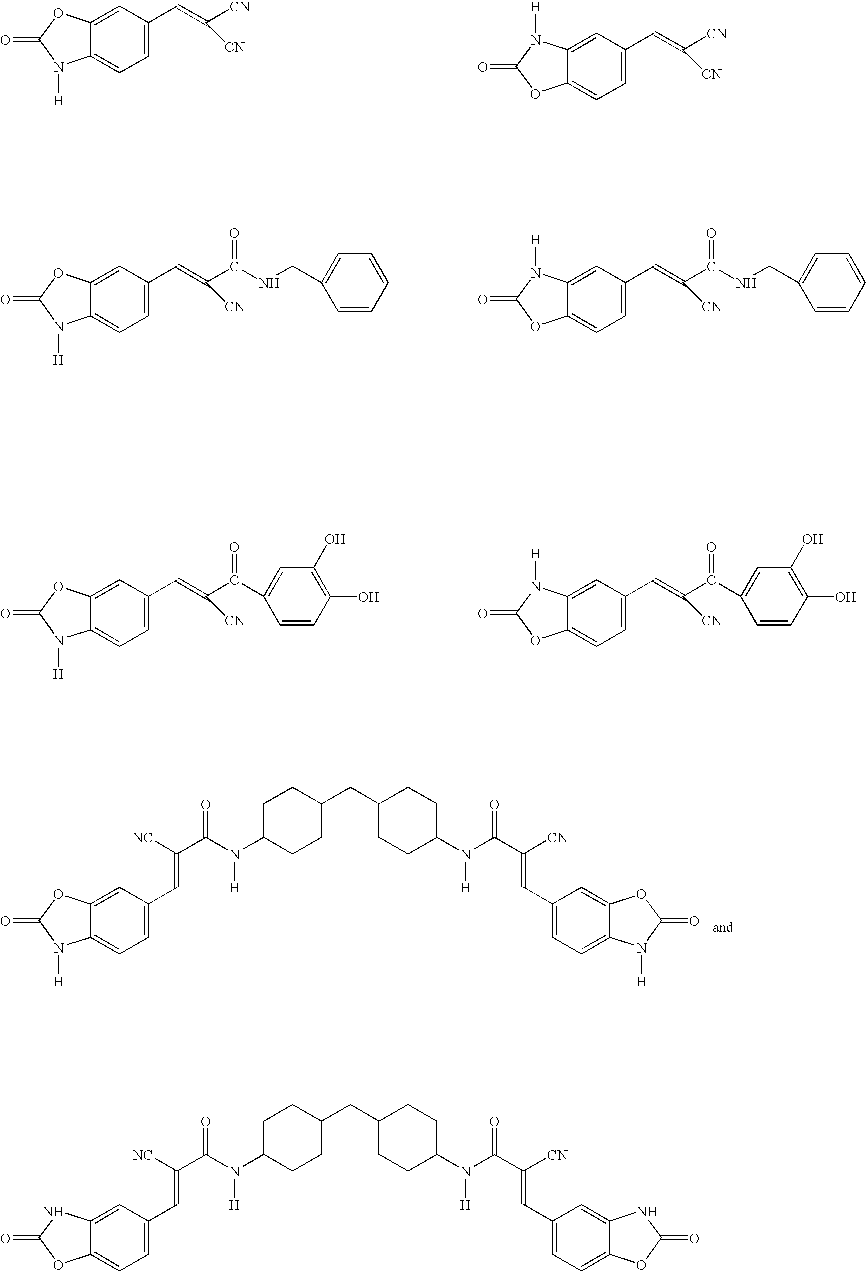

In one embodiment, the protein tyrosine kinase is a receptor protein tyrosine kinase (RTK). In another embodiment, the receptor protein tyrosine kinase is selected from the group consisting of a platelet-derived growth factor receptor (PDGFr), a fibroblast growth factor receptor (FGF), a hepatocyte growth factor receptor (HGFr), an insulin receptor, an insulin-like growth factor-1 receptor (IGF-1r), a nerve growth factor receptor (NGF), a vascular endothelial growth factor receptor (VEGFr), and a macrophage colony stimulating factor receptor (M-CSFr).

### BRIEF DESCRIPTION OF THE DRAWINGS

The present invention will be understood and appreciated more fully from the following detailed description taken in conjunction with the appended drawings, in which:

FIG. 1: Shows in schematic form (Scheme 1) a process for synthesizing Class I Para Isomer catechol bioisosteres.

FIG. 2: Shows in schematic form (Scheme 2) a process for synthesizing Class I Meta Isomer catechol bioisosteres.

FIG. 3: Shows examples of Class I catechol bioisosteres of the present invention.

FIG. 4: Shows in schematic form (Scheme 3) a process for synthesizing Class II Para Isomer catechol bioisosteres.

FIG. 5: Shows in schematic form (Scheme 4) a process for synthesizing Class II Meta Isomer catechol bioisosteres.

FIG. 6: Shows examples of Class II catechol bioisosteres of the present invention.

### DETAILED DESCRIPTION OF THE PRESENT INVENTION

The present invention provides catechol bioisostere compounds which are potent inhibitors of protein tyrosine kinases (PTKs). The present invention further provides methods of inhibiting PTKs, for example receptor protein tyrosine kinases (RTKs), comprising administering the catechol bioisosteres. The catechol bioisostere compounds are useful in treating or preventing PTK-related disease states, particularly protein tyrosine kinase related disorders which are associated with defects in signaling pathways mediated by PTKs.

As contemplated herein, the compounds of the present invention are designed to mimic certain tyrphostins of the benzylidene malononitrile chemical class which contain the oxidation-sensitive catechol pharmacophore. Applicants have surprisingly found that substitution of the catechol pharmacophore with a benzoxazolone moiety results in new compounds, designated ‘catechol bioisosteres’ which, like tyrphostins, are found to be potent inhibitors of protein tyrosine kinases.

The bioisostere compounds provided by the present invention are represented by the general structure of formula 1: 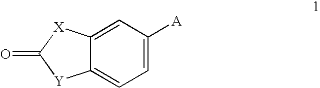
wherein X and Y are independently NR₁ or O, wherein R₁ is H or alkyl; A is a group represented by the formula: 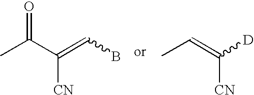
   B is phenyl which is unsubstituted or substituted by one or more OR₂ or COOR₃ wherein R₂ and R₃ are independently hydrogen or alkyl, or B is a group represented by the formula: 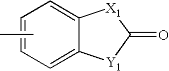
wherein X₁ and Y₁ are independently NR₁ or O, wherein R₁ is H or alkyl; D is CN; C(═O)R₄ wherein R₄ is an alkyl, aralkyl or aryl which is unsubstituted or substituted by one or more OR₅, wherein R₅ is hydrogen or alkyl; or C(═O)NR₆R₇ wherein R₆ and R₇ are independently hydrogen or an optionally substituted alkyl, aralkyl or aryl.

The following is a list of some of the definitions of the chemical groups used in the present disclosure:

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl.

An “aryl” group refers to an aromatic group having at least one carbocyclic aromatic group or heterocyclic aromatic group, which may be unsubstituted or substituted by one or more groups selected from halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxy” group refers to an OH group. An “alkoxy” group refers to an —O-alkyl group wherein alkyl is as defined above. A “thio” group refers to an —SH group. An “alkylthio” group refers to an —SR group wherein R is alkyl as defined above.

An “amino” group refers to an —NH₂ group. An alkylamino group refers to an —NHR group wherein R is alkyl is as defined above. A dialkylamino group refers to an —NRR′ group wherein R and R′ are alkyl as defined above.

An “amido” group refers to an —CONH₂ group. An alkylamido group refers to an —CONHR group wherein R is alkyl is as defined above. A dialkylamido group refers to an —CONRR′ group wherein R and R′ are alkyl as defined above.

An “aralkyl” group refers to an alkyl bound to an aryl, wherein alkyl and aryl are as defined above. An example of an aralkyl group is a benzyl group.

The bioisostere compounds of the present invention are broadly classified into two classes, namely Class I and Class II, as defined herein.

Class I Compounds:

Class I compounds are represented by the general structure of formula 2: 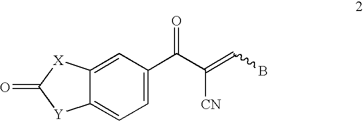
wherein X and Y are independently NR₁ or O, wherein R₁ is H or alkyl; B is phenyl which is unsubstituted or substituted by one or more OR₂ or COOR₃ wherein R₂ and R₃ are independently hydrogen or alkyl, or B is a group represented by the formula: 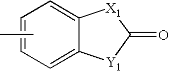
wherein X₁ and Y₁ are independently NR₁ or O, wherein R₁ is H or alkyl.

In one embodiment, X and Y are O. In another embodiment, X is O and Y is NR₁. In another embodiment, X is O and Y is NH. The Class I compounds wherein X is O are designated herein as “Class I Para Isomers”.

In one embodiment, X and Y are NR₁. In another embodiment, X and Y are NH. In another embodiment, X is NR₁ and Y is NH. In another embodiment, X is NH and Y is NR₁. In another embodiment, X is NR₁ and Y is O. In another embodiment, X is NH and Y is O. The Class I compounds wherein X is NR₁ or NH are designated as “Class I Meta Isomers”.

In one embodiment, the Class I compounds are represented by the structure of formula 3, wherein X and Y are as defined above and R₈, R₉ are independently hydroxy, alkoxy or COOH,

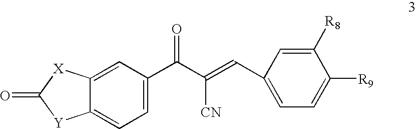

In one embodiment, R₈ and R₉ are both hydroxy. In another embodiment, R₈ is hydroxy and R₉ is methoxy. In another embodiment, R₈ is methoxy and R₉ is hydroxy. In another embodiment, R₈ is COOH and R₉ is hydroxy. In another embodiment, R₈ is hydroxy and R₉ is COOH.

In another embodiment, the class I compounds are represented by the structure of formula 4.

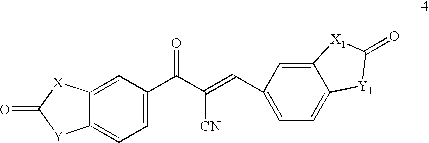

In one embodiment, X₁ and Y₁ are NR₁. In another embodiment, X₁ and Y₁ are NH. In another embodiment, X₁ is NR₁ and Y₁ is NH. In another embodiment, X₁ is NH and Y₁ is NR₁. In another embodiment, X₁ and Y₁ are O. In another embodiment, X₁ is O and Y₁ is NR₁. In another embodiment, X₁ is NR₁ and Y₁ is O. In another embodiment, X₁ is O and Y₁ is NH. In another embodiment, X₁ is NH and Y₁ is O.

In one embodiment, X, X₁, Y and Y₁ are O. In another embodiment, X, X₁, Y and Y₁ are NR₁. In another embodiment, X, X₁, Y and Y₁ are NH. In another embodiment, X and Y are O and X₁ and Y₁ are NR₁. In another embodiment, X and Y are O and X₁ and Y₁ are NH. In another embodiment, X and Y are NR₁ and X₁ and Y₁ are O. In another embodiment, X and Y are NH and X₁ and Y₁ are O.

In one embodiment, X and X₁ are NR₁ and Y and Y₁ are O. In another embodiment, X and X₁ are O are and Y and Y₁ are NR₁. In another embodiment, X and Y₁ are NR₁ and X₁ and Y are O. In another embodiment, X and Y₁ are O and X₁ and Y are NR₁.

In one embodiment, X and X₁ are NH and Y and Y₁ are O. In another embodiment, X and X₁ are O and Y and Y₁ are NH. In another embodiment, X and Y₁ are NH and X₁ and Y are O. In another embodiment, X and Y₁ are O and X₁ and Y are NH.

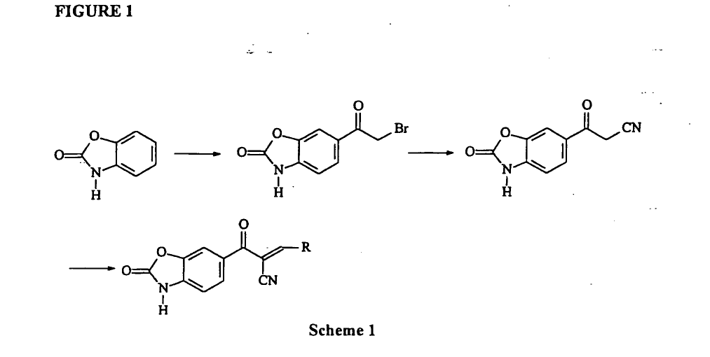

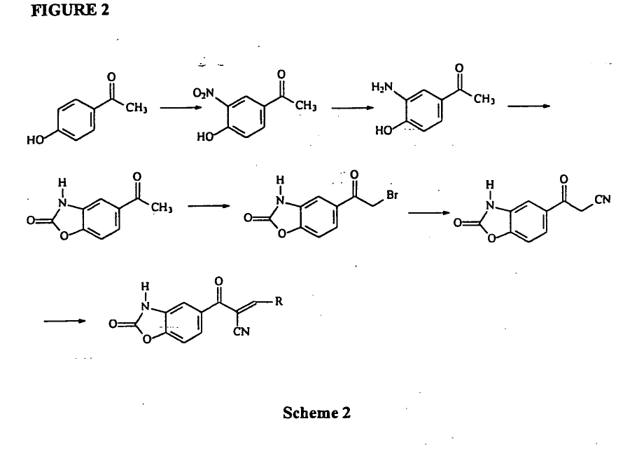

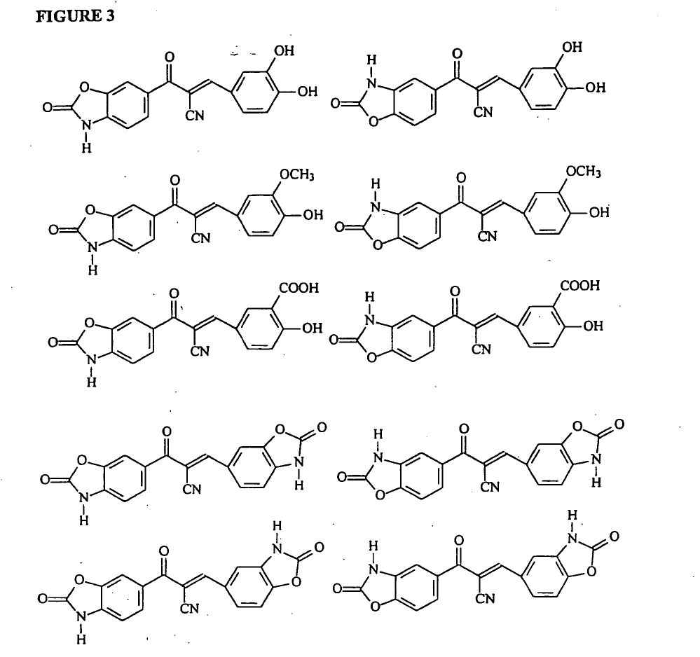

The “Class I Para Isomer” compounds are synthesized as detailed in Scheme I (FIG. 1) and in the Experimental Details Section. The “Class I Meta Isomer” compounds are synthesized as detailed in Scheme 2 (FIG. 2) and in the Experimental Details Section. Examples of Class I compounds are provided in FIG. 3 and in the Experimental Details Section.

Class I Compounds:

Class II compounds are represented by the general formula 5, wherein D is CN; C(═O)R₄ wherein R₄ is an alkyl, aralkyl or aryl which is unsubstituted or substituted by one or more OR₅, wherein R₅ is hydrogen or alkyl; or C(═O)NR₆R₇ wherein R₆ and R₇ are independently hydrogen or an optionally substituted alkyl, aralkyl or aryl.

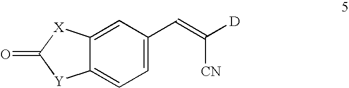

In one embodiment, X and Y are O. In another embodiment, X is O and Y is NR₁. In another embodiment, X is O and Y is NH. The Class II compounds wherein X is O are designated herein as “Class II Para Isomers”.

In one embodiment, X and Y are NR₁. In another embodiment, X and Y are NH. In another embodiment, X is NR₁ and Y is NH. In another embodiment, X is NH and Y is NR₁. In another embodiment, X is NR₁ and Y is O. In another embodiment, X is NH and Y is O. The Class II compounds wherein X is NR₁ or NH are designated as “Class II Meta Isomers”.

In one embodiment, D is CN. In another embodiment, D is C(═O)R₄ wherein R₄ is an alkyl, aralkyl or aryl which is unsubstituted or substituted by one or more OR₅, wherein R₅ is hydrogen or alkyl. In one embodiment, R₄ is phenyl. In another embodiment, R₄ is phenyl substituted by one OH. In another embodiment, R₄ is phenyl substituted by two OH groups. In another embodiment, R₄ is phenyl substituted by two OH groups at the 3 and 4 positions.

In one embodiment, C(═O)NR₆R₇ wherein R₆ and R₇ are independently hydrogen or an optionally substituted alkyl, aralkyl or aryl. In one embodiment, R₆ is hydrogen and R₇ is an optionally substituted alkyl, aralkyl or aryl. In another embodiment, R₇ is an aralkyl. In another embodiment, R₇ is benzyl. In another embodiment, R₇ is cyclohexyl. In another embodiment, R₇ is a substituted cyclohexyl.

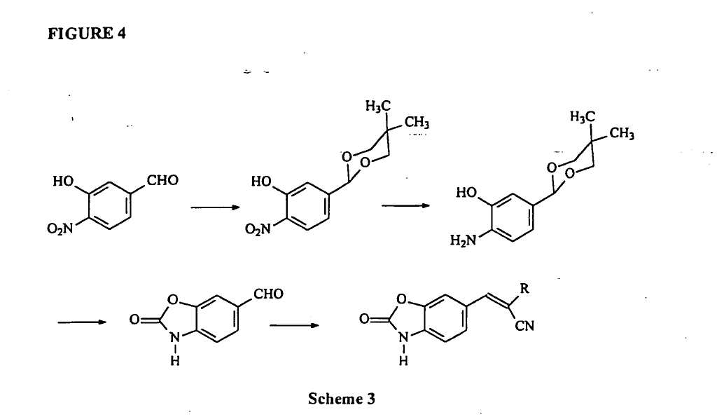

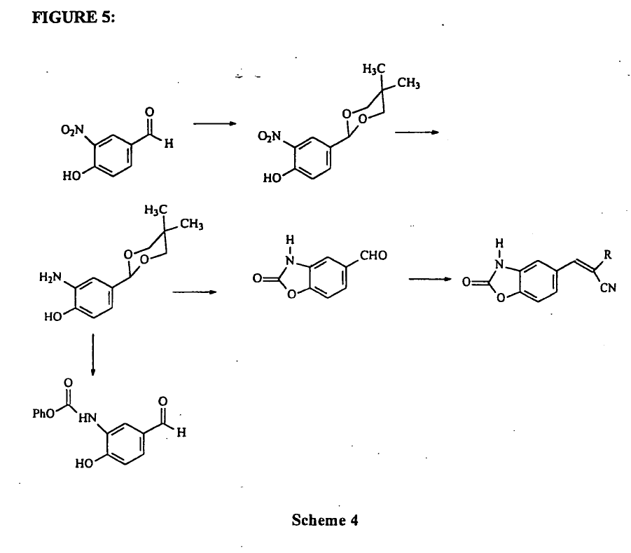

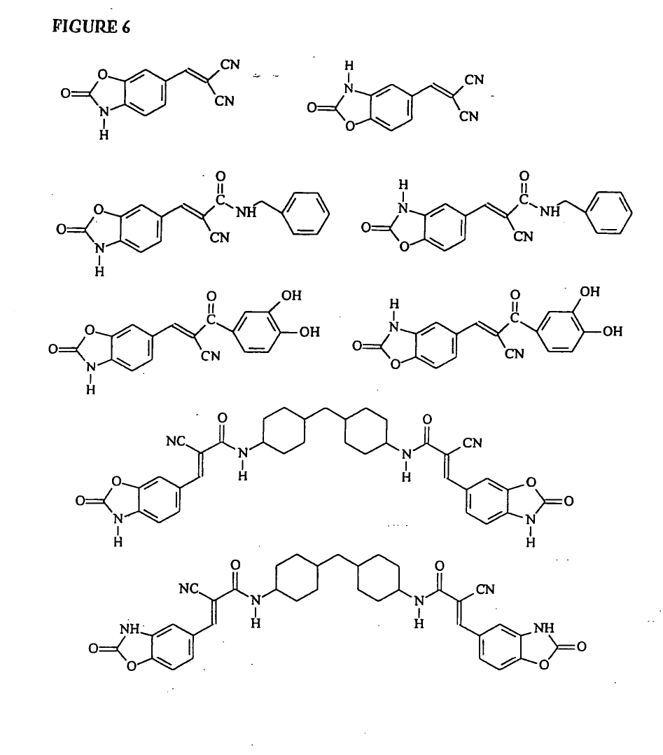

The “Class II Para Isomer” compounds are synthesized as detailed in Scheme 3 (FIG. 4) and in the Experimental Details Section. The “Class II Meta Isomer” compounds are synthesized as detailed in Scheme 4 (FIG. 5) and in the Experimental Details Section. Examples of Class II compounds are provided in FIG. 6 and in the Experimental Details Section.

The present invention provides compounds and compositions effective at inhibiting protein tyrosine kinases. These compounds and compositions are potentially useful in the treatment of a diseases associated with altered or abnormal activity of protein tyrosine kinases such as enhanced activity of protein tyrosine kinases. As contemplated herein, the present invention encompasses the use of catechol bioisostere compounds of formulas 1-5 and their isomers, pharmaceutically acceptable salts and hydrates thereof. In addition, the present invention encompasses the use of mixtures of the compounds or their isomers, pharmaceutically acceptable salts and hydrates thereof. An isomer of the compound includes, but is not limited to optical isomers, structural isomers, conformational isomers and analogs, and the like.

In one embodiment, this invention encompasses the use of different optical isomers of the compounds of any of formulas 1-5 in inhibiting protein tyrosine kinases. It will be appreciated by those skilled in the art that the compounds of the present invention may contain at least one chiral center. Accordingly, the compounds used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, which form possesses properties useful in the treatment protein tyrosine kinase related disorders, as defined herein. In one embodiment, the compounds of the present invention are the pure (R)-isomers. In another embodiment, the compounds of the present invention are the pure (S)-isomers. In another embodiment, the compounds of the present invention are a mixture of the (R) and the (S) isomers. In another embodiment, the compounds of the present invention are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

In one embodiment, this invention encompasses the use of different structural isomers of the catechol bioisostere compounds of the present invention. It will be appreciated by those skilled in the art that the compounds of the present invention may exist as the (Z) or the (E) isomers. The invention encompasses pure (Z)- and (E)-isomers of the compounds defined herein and mixtures thereof.

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

For use in medicine, the salts of the compounds will be pharmaceutically acceptable salts. Pharmaceutically acceptable salts include the acid addition salts which are formed by the reaction of free amino groups with inorganic acids such as, for example, hydrochloric or phosphoric acids, or such organic acids as acetic, oxalic, tartaric, mandelic, and the like. Other salts may, however, be useful in the preparation of the compounds according to the invention or of their pharmaceutically acceptable salts. Suitable pharmaceutically acceptable salts of the compounds of this invention include acid addition salts, which may, for example, be formed by mixing a solution of the compound according to the invention with a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

This invention further includes derivatives of the catechol bioisostere compounds of any of formulas 1-5. The term “derivative” includes but is not limited to ether derivatives, acid derivatives, amide derivatives, acid derivatives, ester derivatives and the likes. In addition, this invention further includes hydrates of the compounds defined herein. The term “hydrate” includes but is not limited to hemihydrate, monohydrate, dihydrate, trihydrate and the like.

The present invention further provides pharmaceutical compositions comprising any of the compounds represented by formulas 1-5, and a pharmaceutically acceptable carrier or excipient.

As used herein, “pharmaceutical composition” means therapeutically effective amounts of the compounds of the present invention, together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or Lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

Also comprehended by the invention are particulate compositions coated with polymers (e.g., poloxamers or poloxamines). Other embodiments of the compositions of the invention incorporate particulate forms, protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intracranially or intratumorally.

Further, as used herein “pharmaceutically acceptable carriers” are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

Other embodiments of the compositions of the invention incorporate particulate forms, protective coatings, protease inhibitors or permeation enhancers for various routes of administration, including parenteral, pulmonary, nasal and oral.

Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14: 201 (1987); Buchwald et al., Surgery 88: 507 (1980); Saudek et al., N. Engl. J. Med. 321: 574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Preferably, a controlled release device is introduced into a subject in proximity to the site of inappropriate immune activation or a tumor. Other controlled release systems are discussed in the review by Langer (Science 249: 1527-1533 (1990).

The pharmaceutical preparation can comprise one or more of the compounds of formulas 1-5 alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the selective androgen receptor modulator can be administered to a subject by, for example, subcutaneous implantation of a pellet; in a further embodiment, the pellet provides for controlled release of selective androgen receptor modulator over a period of time. The preparation can also be administered by intravenous, intraarterial, or intramuscular injection of a liquid preparation, oral administration of a liquid or solid preparation, or by topical application. Administration can also be accomplished by use of a rectal suppository or a urethral suppository.

The pharmaceutical preparations of the invention can be prepared by known dissolving, mixing, granulating, or tablet-forming processes. For oral administration, the selective androgen receptor modulators or their physiologically tolerated derivatives such as salts, esters, N-oxides, and the like are mixed with additives customary for this purpose, such as vehicles, stabilizers, or inert diluents, and converted by customary methods into a suitable form for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, or with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the compounds of the present invention or their physiologically tolerated derivatives such as salts, hydrates and the like are converted into a solution, suspension, or emulsion, if desired with the substances customary and suitable for this purpose, for example, solubilizers or other auxiliaries. Examples are sterile liquids such as water and oils, with or without the addition of a surfactant, and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycols are preferred liquid carriers, particularly for injectable solutions.

The preparation of pharmaceutical compositions which contain an active component is well understood in the art. Typically, such compositions are prepared as aerosols of the polypeptide delivered to the nasopharynx or as injectables, either as liquid solutions or suspensions, however, solid forms suitable for solution in, or suspension in, liquid prior to injection can also be prepared. The preparation can also be emulsified. The active therapeutic ingredient is often mixed with excipients that are pharmaceutically acceptable and compatible with the active ingredient. Suitable excipients are, for example, water, saline, dextrose, glycerol, ethanol, or the like and combinations thereof.

In addition, if desired, the composition can contain minor amounts of auxiliary substances such as wetting or emulsifying agents, pH buffering agents, which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

For topical administration to body surfaces using, for example, creams, gels, drops, and the like, the compounds of the present invention or their physiologically tolerated derivatives such as salts, hydrates, and the like are prepared and applied as solutions, suspensions, or emulsions in a physiologically acceptable diluent with or without a pharmaceutical carrier.

In another embodiment, the active compound can be delivered in a vesicle, in particular a liposome (see Langer, Science 249: 1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

The present invention further provides a method of inhibiting a protein tyrosine kinase (PTK) comprising contacting the PTK with an effective inhibitory amount of a compound represented by any of formulas 1-5.

The present invention further provides a method of treating or preventing a protein tyrosine kinase (PTK) in a subject comprising the step of administering to the subject a therapeutically effective amount of any of the compounds represented by formulas 1-5. In another embodiment, the method comprises administering to the subject a pharmaceutical composition comprising a therapeutically effective amount of any of the compounds represented by formulas 1-5 and a pharmaceutically acceptable excipient.

The present invention further provides a method of inhibiting a protein tyrosine kinase (PTK) related disorder in a subject comprising the step of administering to the subject a therapeutically effective amount of any of the compounds represented by formulas 1-5. In another embodiment, the method comprises administering to the subject a pharmaceutical composition comprising a therapeutically effective amount of any of the compounds represented by formulas 1-5; and a pharmaceutically acceptable excipient.

A “protein tyrosine kinase” (PTK) is a protein belonging to a family of enzymes which transfer the γ-phosphate of ATP to the side chain of tyrosine residues on substrate proteins. PTKs are involved in a variety of key cellular processes, including signal transduction and growth regulation. A protein tyrosine kinase, as used herein, refers to a receptor tyrosine kinase (RTK) as well as a cellular tyrosine kinase (CTK or non-receptor tyrosine kinase). Thus the compounds of the present invention are effective at inhibiting both receptor and non-receptor protein tyrosine kinases.

A cellular tyrosine kinase (CTK or non-receptor tyrosine kinase) is an intracellular protein which takes part in signal transduction within the cell, including signal transduction to the nucleus. Examples of CTKs are the Src family of oncoproteins. A receptor tyrosine kinases (RTK) is a transmembrane protein which participates in transmembrane signaling pathways. The predominant biological activity of some receptor tyrosine kinases is the stimulation of cell growth and proliferation, while other receptor tyrosine kinases are involved in arresting growth and promoting differentiation. RTKs include the receptors for platelet-derived growth factor (PDGF), fibroblast growth factor (FGF), hepatocyte growth factor (HGF), insulin, insulin-like growth factor-1 (IGF-1), nerve growth factor (NGF), vascular endothelial growth factor (VEGF), and macrophage colony stimulating factor (M-CSF).

The term “protein tyrosine kinase related disorder” as used herein refers to a disorder characterized by abnormal or altered PTK activity. Abnormal or altered activity further refers to either (i) overexpression of PTK in cells which do not normally express PTKs; (ii) increased PTK expression leading to unwanted cell proliferation, differentiation and/or growth; or, (iii) decreased PTK expression leading to unwanted reductions in cell proliferation, differentiation and/or growth. Over-activity of PTKs refers to either amplification of the gene encoding a particular PTK or production of a level of PTK activity which can correlate with a cell proliferation, differentiation and/or growth. Over-activity can also be the result of ligand independent or constitutive activation as a result of mutations such as deletions of a fragment of a PTK responsible for ligand binding.

Thus, in one embodiment, the present invention is directed to catechol bioisostere-containing preparations, which modulate PTK activity signal transduction by affecting the enzymatic activity of the protein tyrosine kinases thereby interfering with the signal transduction pathways mediated by such proteins.

Examples of protein tyrosine kinase related disorders are cell proliferative disorders, metabolic disorders or fibrotic disorders.

Examples of cell proliferative disorders which are mediated by protein tyrosine kinases are cancer, blood vessel proliferative disorders, and mesangia cell proliferative disorders.

Cancer is a disorder in which a population of cells has become, in varying degrees, unresponsive to the control mechanisms that normally govern proliferation and differentiation. Cancer refers to various types of malignant neoplasms and tumors, including metastasis to different sites. Nonlimiting examples of cancers which can be treated by the catechol bioisostere compounds of formulas 1-5 are brain, ovarian, colon, prostate, kidney, bladder, breast, lung, oral and skin cancers which exhibit altered activity of PTK. Specific examples of cancers which the compounds of the present invention are effective at treating or preventing are: adenocarcinoma, adrenal gland tumor, ameloblastoma, anaplastic tumor, anaplastic carcinoma of the thyroid cell, angiofibroma, angioma, angiosarcoma, apudoma, argentaffinoma, arrhenoblastoma, ascites tumor cell, ascitic tumor, astroblastoma, astrocytoma, ataxia-telangiectasia atrial myxoma, basal cell carcinoma, benign tumor, bone cancer, bone tumor, brainstem glioma, brain tumor, breast cancer, Burkitt's lymphoma, carcinoma, cerebellar astrocytoma, cervical cancer, cherry angioma, cholangiocarcinoma, a cholangioma, chondroblastoma, chondroma, chondrosarcoma, chorioblastoma, choriocarcinoma, colon cancer, common acute lymphoblastic leukaemia, craniopharyngioma, cystocarcinoma, cystofibroma, cystoma, cytoma, ductal carcinoma in situ, ductal papilloma, dysgerminoma, encephaloma, endometrial carcinoma, endothelioma, ependymoma, epithelioma, erythroleukaemia, Ewing's sarcoma, extra nodal lymphoma, feline sarcoma, fibroadenoma, fibrosarcoma, follicular cancer of the thyroid, ganglioglioma, gastrinoma, glioblastoma multiforme, glioma, gonadoblastoma, haemangioblastoma, haemangioendothelioblastoma, haemangioendothelioma, haemangiopericytoma, haematolymphangioma, haemocytoblastoma, haemocytoma, hairy cell leukaemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angiofibroma, Kaposi sarcoma, kidney tumour, large cell lymphoma, leukemia, chronic leukemia, acute leukemia, lipoma, liver cancer, liver metastases, Lucke carcinoma, lymphadenoma, lymphangioma, lymphocytic leukaemia, lymphocytic lymphoma, lymphocytoma, lymphoedema, lymphoma, lung cancer, malignant mesothelioma, malignant teratoma, mastocytoma, medulloblastoma, melanoma, meningioma, mesothelioma, metastatic cancer, Morton's neuroma, multiple myeloma, myeloblastoma, myeloid leukemia, myelolipoma, myeloma, myoblastoma, myxoma, nasopharyngeal carcinoma, nephroblastoma, neuroblastoma, neurofibroma, neurofibromatosis, neuroglioma, neuroma, non-Hodgkin's lymphoma, oligodendroglioma, optic glioma, osteochondroma, osteogenic sarcoma, osteosarcoma, ovarian cancer, Paget's disease of the nipple, pancoast tumor, pancreatic cancer, phaeochromocytoma, pheochromocytoma, plasmacytoma, primary brain tumor, progonoma, prolactinoma, renal cell carcinoma, retinoblastoma, rhabdomyosarcoma, rhabdosarcoma, solid tumor, sarcoma, secondary tumor, seminoma, skin cancer, small cell carcinoma, squamous cell carcinoma, strawberry haemangioma, T-cell lymphoma, teratoma, testicular cancer, thymoma, trophoblastic tumor, tumourigenic, vestibular schwannoma, Wilm's tumor, or a combination thereof.

Blood vessel proliferative disorders refer to antiogenic and vasculogenic disorders generally resulting in abnormal proliferation of blood vessels. The formation and spreading of blood vessels, or vasculogenesis and angiogenesis, respectively, play important roles in a variety of physiological processes such as embryonic development, corpus luteum formation, wound healing and organ regeneration, as well as a pivotal role in cancer development. Other examples of blood vessel proliferation disorders include arthritis and ocular diseases such as diabetic retinopathy. Other examples are restenosis, retinopathies and atherosclerosis.

Mesangial cell proliferative disorders refer to disorders brought about by abnormal proliferation of mesangial cells. Mesangial proliferative disorders include various human renal diseases such as glomerulonephritis, diabetic nephropathy, malignant nephrosclerosis, thrombic microangiopathy syndromes, transplant rejection and glomerulopathies. In this regards, PDGFR has been implicated in the maintenance of mesangial cell proliferation.

Metabolic disorders that are implicated with abnormal PTK activity include psoriasis, diabetes mellitus, wound healing, inflammation and neurodegenerative diseases. For example, EGFR has been indicated in corneal and dermal wound healing. Defects in the Insulin-R and IGF-1R receptor are indicated in type-II diabetes mellitus.

Fibrotic disorders refer to the abnormal formation of extracellular matrices. Examples of fibrotic disorders include hepatic cirrhosis and mesangial cell proliferative disorders. Hepatic cirrhosis is characterized by the increase in extracellular matrix constituents resulting in the formation of a hepatic scar.

The term “treating” as used herein refers to abrogating, inhibiting, slowing or reversing the progression of a disease, ameliorating clinical symptoms of a disease or preventing the appearance of clinical symptoms of a disease. The term “preventing” is defined herein as barring a subject from acquiring a disorder or diseases in the first place.

The term “administering” as used herein refers to a method for bringing a catechol bioisostere compound of the present invention and a target protein tyrosine kinase together in such a manner that the tyrphostin can affect the catalytic activity of the tyrosine kinase directly; i.e. by interacting with the kinase itself, or indirectly; i.e. by interacting with another molecule on which the catalytic activity of the enzyme is dependent. As used herein, administration can be accomplished in vitro, i.e. in a test tube, or in vivo, i.e. in cells or tissues of living organisms, for example humans. In one embodiment, the present invention encompasses administering the compounds of the present invention to a subject.

The term “contacting” as used herein refers to bringing into contact the protein tyrosine kinase and the compounds defined herein, under in vivo conditions or in vitro conditions as defined above.

The term “therapeutically effective amount refers to the amount of a compound being administered which relieves to some extent one or more of the symptoms of the disorder being treated. Therapeutic effective doses for the catechol bioisosteres described herein can be estimated initially from cell culture and/or an animal model. A dose can be formulated in an animal model, and this dose can be used to more precisely determine useful doses in humans.

The term “effective inhibitory amount refers to the amount of a compound being administered which inhibits to some extent the protein tyrosine kinase with which it is contacted.

In one embodiment, the compounds which are useful in inhibiting PTKs and PTK related cell proliferative disorder are selected from the group consisting of:

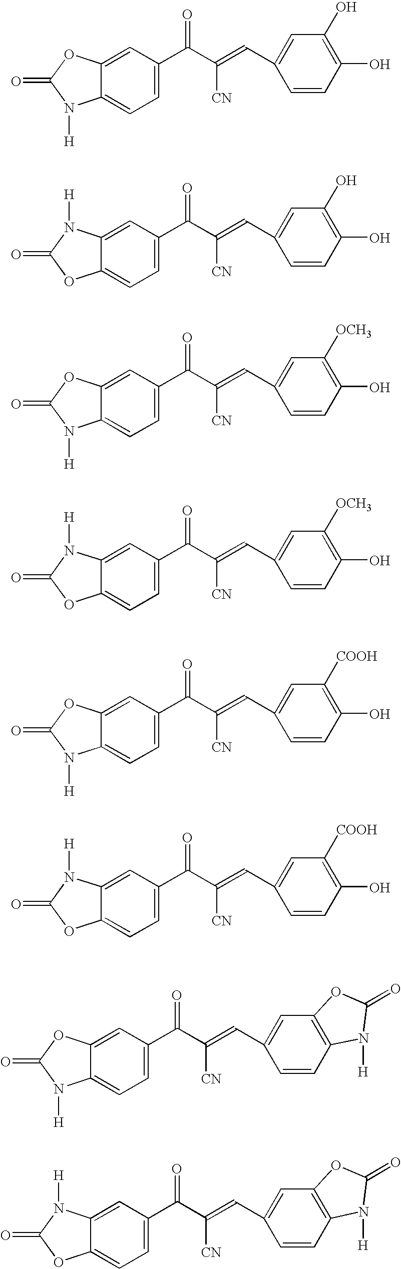

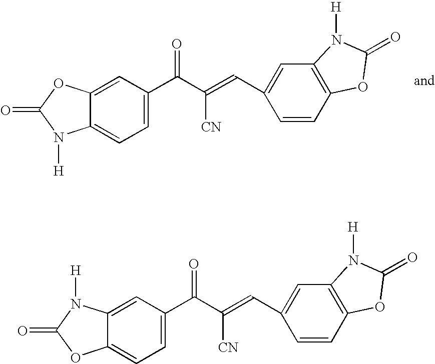

In another embodiment, the compounds which are useful in inhibiting PTKs and PTK related cell proliferative disorder are selected from the group consisting of:

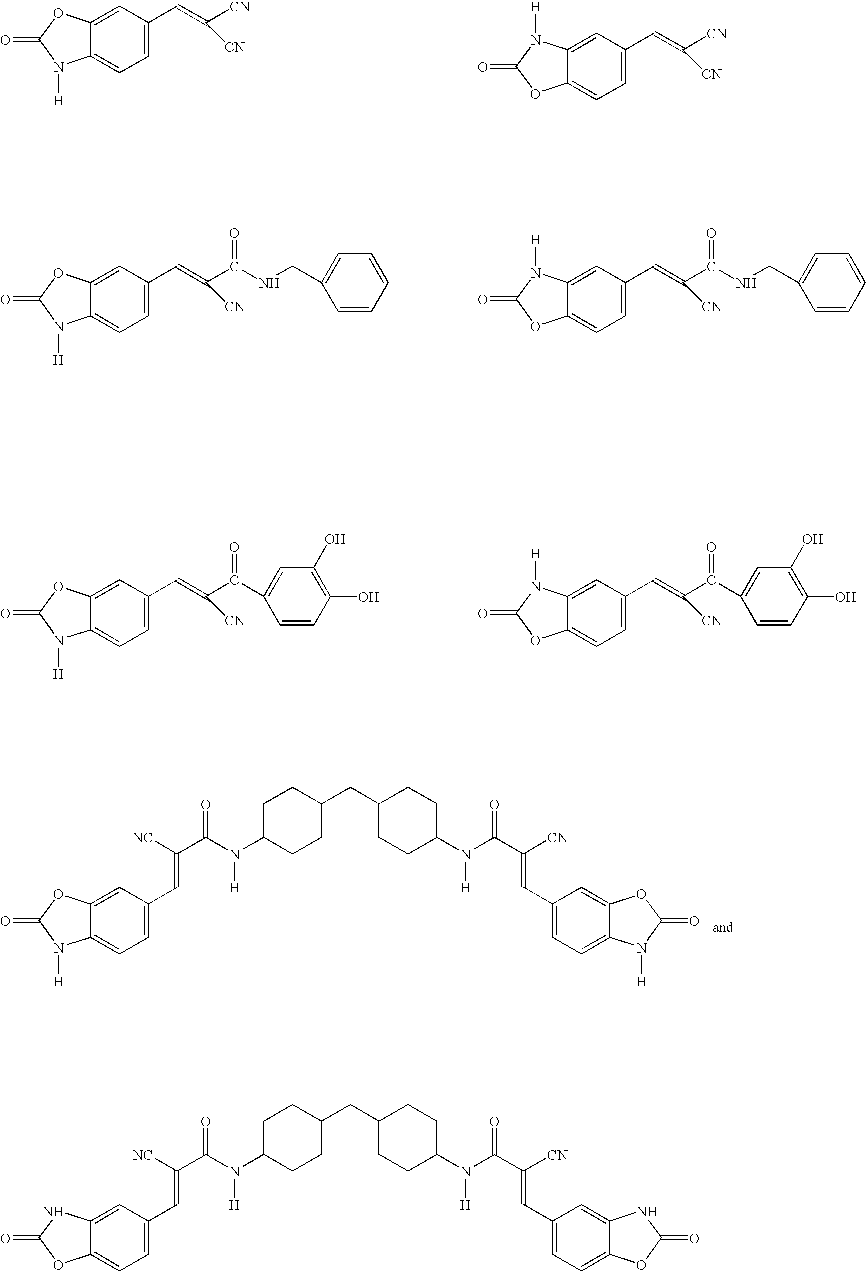

In one embodiment, the protein tyrosine kinase is a receptor protein tyrosine kinase (RTK). In another embodiment, the receptor protein tyrosine kinase is selected from the group consisting of a platelet-derived growth factor receptor (PDGFr), a fibroblast growth factor receptor (FGF), a hepatocyte growth factor receptor (HGFr), an insulin receptor, an insulin-like growth factor-1 receptor (IGF-1r), a nerve growth factor receptor (NGF), a vascular endothelial growth factor receptor (VEGFr), and a macrophage colony stimulating factor receptor (M-CSFr).

The following examples are presented in order to more fully illustrate the preferred embodiments of the invention. They should in no way, however, be construed as limiting the broad scope of the invention.

### EXPERIMENTAL DETAILS SECTION

### Example 1

Preparation of Catechol Bioisosteres—Class I-Para Isomers

Para Isomers of Class I Catechol Bioisosteres were prepared according to Scheme 1 (FIG. 1).

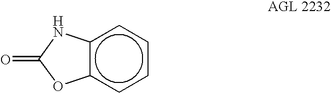

11 gr, 0.19 M, phenylene diamine and 8.4 gr sodium bicarbonate in 50 ml water and 50 ml ethanol were stirred 0.5 hour at room temperature. 15.6 ClCOOPh was added in portions followed by 4 gr NaOH. After 5 minutes 15 ml HCl in 50 ml water was added. The pasty precipitate was filtered and dried to give 8.5 gr, 62% yield, light-brown solid, mp-125°. NMR CDCl₃ δ7.23-7.09 (4H,m).

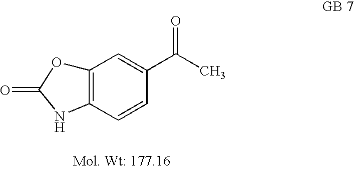

3 gr (22 mM) AG 2232 (benzoxazolone), 45 gr poly phosphoric acid (PPA) and 1950 μl 33 mM, acetic acid were heated to 125° C. for 4.5 hours. The reaction mixture was poured into ice, stirred for 0.5 h and filtered, and the precipitate was dissolved in 10 ml acetone and 100 ml ethyl acetate. 100 ml water was added and the product was extracted to 3*100 ml ethyl acetate, then subsequently evaporated, followed by chromatography on silica 35-70. 460 mg GB 7 was eluted with 25% ethyl acetate in petroleum ether to yield 11.3%, Melting point=227° C. ¹H-NMR (CDCl₃, [symbol] ppm) 2.61 (s,3H), 7.12(d,1H, J=8.1 Hz), 7.83 (d, 1H, J=1.3 Hz), 7.86 (dd, 1H, J=1.5,8.2).

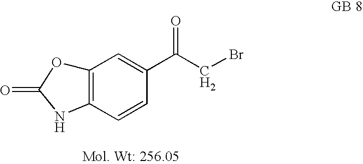

The method is based on a similar reaction described by Yous et. Al J. Org. Chem. 1994, 59, 1574-1576. GB8 (6 bromo-acyl 2(3H)-benzoxazolone) was prepared by adding anhydrous DMF (4.3 ml, 0.067 mol) drop-wise to finely ground AlCl₃ (0.2 mol, 26.65 gr) with stirring under Argon. The mixture was heated to 45° C., and benzoxazolone (0.02 mol, 2.7 gr) and bromoacetylbromide (0.03 mol, 2.65 ml) were added slowly. After 30 minutes the mixture was heated to 95° C. for 4.5 hours, then poured into ice (1 kg), and stirred for 1 hour. The precipitate was filtered and washed with 1 liter of water, then dried and recrystallized from methanol to give (4.6 gr, 0.018 mol) GB 8, yield 90%. Melting point=205° C., ¹H-NMR (acetone-d₆, δ ppm) 4.75 (s,2H), 7.30 (d,1H, J=8.1 Hz), 7.89 (d, 1H, J=1.2 Hz), 7.97 (dd, 1H, J=1.6,8.2). Confirmed by GC-MS.

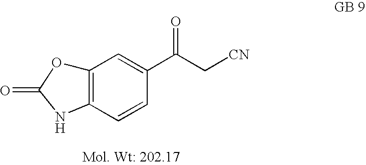

GB8 (7.87 mmol, 2 gr) and KCN (1.02 gr, 15.7 mmol) were dissolved in 10% water/ethanol (400 ml). The mixture was stirred and heated to 45° C. for 1.5 hours, 200 ml of water were then added and the mixture was titrated to pH 6 by addition of HCl. The mixture was stirred for an additional 0.5 hour at room temperature, then brought to pH=7 by adding KOH. The ethanol was evaporated and the product was extracted with 3×200 ml ethyl acetate, then evaporated, followed by chromatography on silica 35-70. GB9 was eluted with 0.6% methanol in dichloromethane to yield 11%, Melting point=200° C., ¹H-NMR (acetone-d₆, δ ppm) 4.58 (s,2H), 7.31 (d,1H, J=8.1 Hz), 7.86 (d, 1H, J=1.6 Hz), 7.92 (dd, 1H, J=1.6,8.2). Confirmed by MS.

The following Class I Para Isomer catechol bioisostere compounds were prepared:

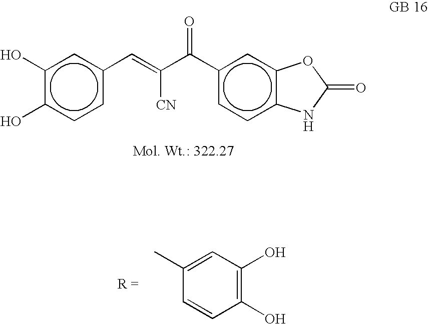

20 mg GB 9, 0.1 mM, 13.6 mg 3.4 dihydroxy benzaldehyde, 0.1 mM and 1.22 β-alanine in 10 ml ethanol were refluxed 4.5 hours then evaporated. Two isomers, GB16-10 and GB 16-11, were cleaned by HPLC using semipreparative RP18 column. Products were eluted by 30% and 36% acetonitrile in water respectively, lyophilized to give 12 mg, GB 16-10 yellow powder, 38% yield, Mp=269° C. and 2 mg, GB 16-11 yellow powder, 5% yield Mp=267° C. GB 16-10 ¹H-NMR (acetone-d₆, δ ppm) 7.02 (d,1H, J=8.3), 7.31 (d,1H, J=8.1), 7.47 (dd, 1H, J=2, 8.3), 7.75 (S, 1H), 7.78 (dd, 1H, J=1.5, 8.1), 7.83 (d, 1H, J=2) 7.98 (S, 1H, vinil). Confirmed by positive MS. GB 16-11 ¹H-NMR (acetone-d₆, δ ppm) 7.02 (d, 1H, J=8.2), 7.40 (d, 1H, J=8.0), 7.47 (dd, 1H, J=2.4, 8.1), 7.61 (d, 1H, J=2), 7.70 (dd, 1H, J=1.6, 8.4), 7.82 (d, 1H, J=2.4) 7.98 (S, 1H, vinyl). Confirmed by positive MS.

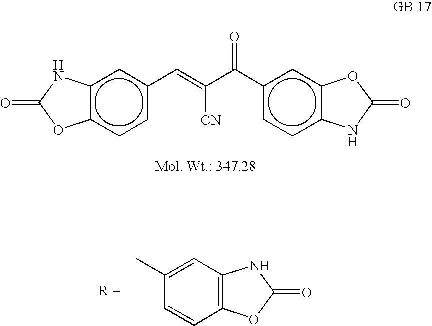

12.4 mg GB 9, 0.06 mM, 10 mg AGL 2253, 0.06 mM and 0.54 mg β-alanine in 10 ml ethanol were refluxed 2 hours then evaporated. The product was cleaned by HPLC using semipreparative RP18 column. GB 17 was eluted by 45% acetonitrile in water, to give 5.12 mg of light-yellow powder, 24% yield. Confirmed by positive MS, Mp=187° C. ¹H-NMR (acetone-d₆, δ ppm) 7.35 (d, 1H, J=8.2), 7.47 (d, 1H, J=8.4), 7.84(m, 3H), 8.06 (d, 1H, J=1.6), 8.17 (S, 1H, vinyl).

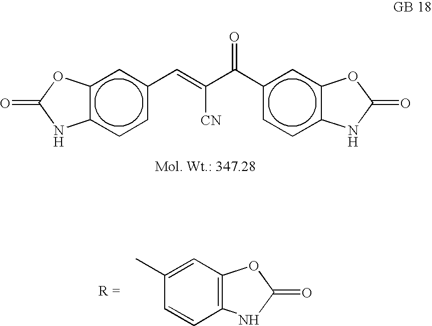

15 mg GB 9, 0.07 mM, 12 mg AGL 2254, 0.07 mM and 0.65 mg β-alanine in 10 ml ethanol were refluxed 4 hours then evaporated. The product was cleaned by HPLC using semipreparative RP18 column. GB 17 was eluted by 39% acetonitrile in water, to give 0.75 mg of light-yellow powder, 3% yield. Mp=182° C. Confirmed by positive MS.

The following compounds can be prepared by similar methods:

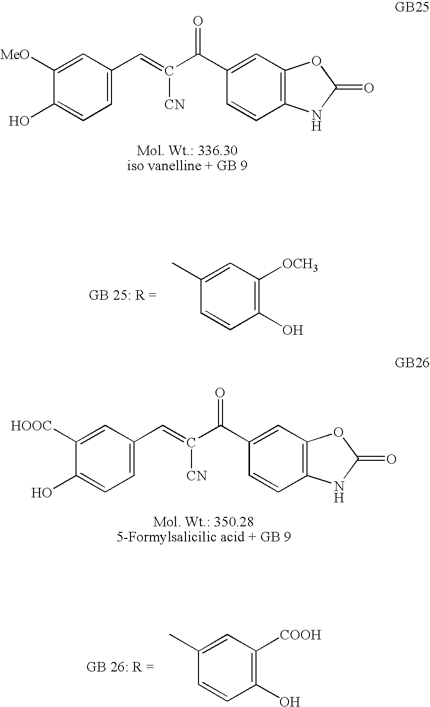

### Example 2

Preparation of Catechol Bioisosteres—Class I-Meta Isomers

Meta Isomers of Class I Catechol Bioisosteres were prepared according to Scheme 2 (FIG. 2). 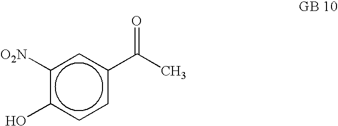
Mol. Wt.: 181.15

27 gr, 0.2 mol of commercial 4-hydroxyacetophenone was nitrated in 100 ml of glacial acetic acid with 25 ml of ION nitric acid under ice cooling and gentle stirring for 6 hours. The reaction mixture was kept overnight at 4° C. while crystallization started. 100 ml water was added, the mixture was cooled in crushed ice for 0.5 h, the precipitate was filtered, washed with water, and dried. The precipitate was recrystallized with ethanol to give 19.6 gr, 54% yield, light-yellow solid, mp=118° C.

¹H-NMR (DMSO-d₆, δ ppm) 2.56 (s 3H) 7.21 (d 1H J=8.7) 8.09 (dd 1, J=2.1,8.7) 8.41 (d 1H, J=2) (Szell T. et al. 1981 J. Chem. Eng. Data 26, 230).

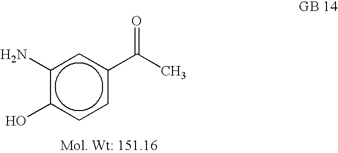

10 gr GB 10, 55 mM was hydrogenated with 10% Pd on active carbon in 150 ml ethanol for 3 h in hydrogen gas. Pd was filtered out, and the mixture was evaporated to give 7.6 gr brown solid, 92% yield, mp=93° C. ¹H-NMR (DMSO-d₆, δ ppm) 2.40 (s 3H) 6.71 (d 1H J=8.1) 7.13 (dd 1, J=2.2,8.2) 7.20 (d 1H, J=2.2).

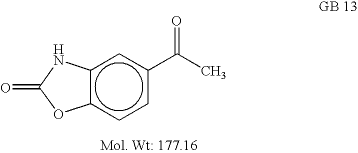

7.6 gr GB 14, 50 mM, 4.2 gr sodium bicarbonate and 7.8 gr ClCOOPh in 150 ml water and 350 ml ethanol were stirred at room temperature for 35 min. 3.75 gr NaOH in 80 ml water was-added and stirred for an additional 30 min (bringing the mixture to pH 12). HCl was added slowly until the reaction mixture reached pH 4, then stirred 10 min and brought back to pH 7 with NaOH 1M. The product was extracted with 3*150 ml ethyl acetate then evaporated. The product was collected in portions by precipitating with water (100 ml ethyl acetate was added mixed with 150 ml water, precipitate 1 was filtered from water phase. Ethyl acetate was evaporated, 150 ml ethanol mixed with 100 ml water were added, precipitate 2 was collected. Most of ethanol was evaporated and mixture was cooled over-night at 4° C., precipitate 3 was collected). Total precipitates gave 4.94 gr, 56% yield of light-brown solid, mp=200° C. ¹H-NMR (DMSO-d₆, δ ppm) 2.58 (s 3H), 7.41 (d 1H J=8.4), 7.57 (d 1H, J=1.7) 7.83 (dd 1, J=1.8, 8.4).

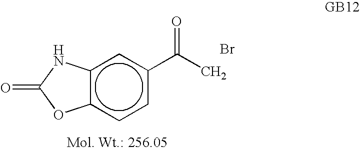

4.94 gr GB13, 28 mM and 12.5 gr finely ground CuBr₂, 56 mM in 150 ml ethyl acetate and 150 ml chloroform was refluxed 15 hours. Reaction mixture was evaporated, 100 ml dichloromethane and 100 ml water added and shaken well. GB 12 was filtered and dried to give 3.9 gr of light-brown solid, 55% yield. Mp=200° C., ¹H-NMR (acetone-d₆, δ ppm) 4.77 (s 2H), 7.39 (d 1H J=8.4), 7.78 (d 1H, J=1.7) 7.92 (dd 1, J=1.8, 8.4). Confirmed by MS.

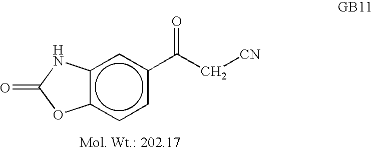

1.024 gr GB13, 4 mM and 0.98 gr NaCN, 20 mM in 100 ml ethanol and 20 ml water were heated to 55° c. for 25 min. Ethanol was evaporated, 150 ml water were added and brought to pH 7 with HCl 1 M (about 5 ml). The product was extracted with 3*100 ml Ethyl acetate (then washed with water) followed by HPLC chromatography using preparative RP18 column. GB 12 was eluted by 25% acetonitrile in water and lyophilized to give 235 mg of white powder, 29% yield Mp=230° C., ¹H-NMR (acetone-d₆, δ ppm) 4.60 (s 2H), 7.40 (d 1H J=8.4), 7.76 (d 1H, J=1.5) 7.87 (dd 1, J=1.8, 8.4). Confirmed by negative MS.

The following Class I Meta Isomer catechol bioisosteres can be prepared:

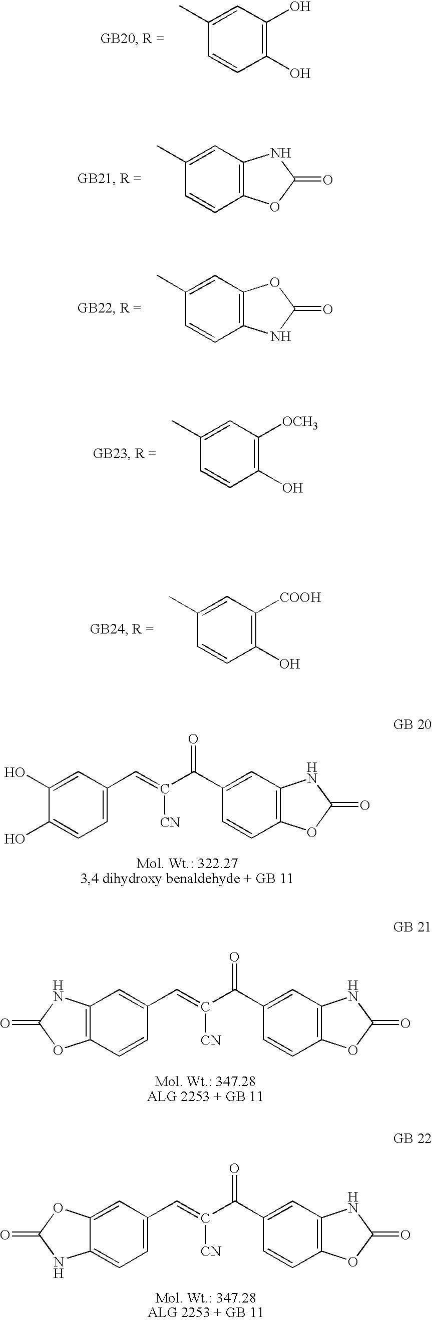

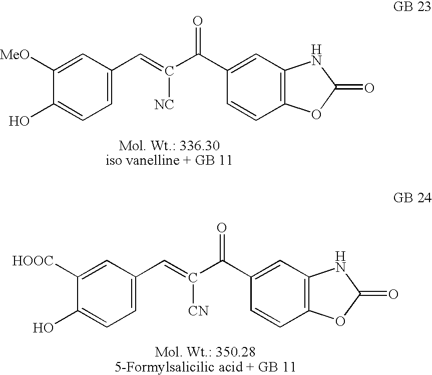

### Example 3

Preparation of Catechol Bioisosteres—Class II-Para Isomers

Para Isomers of Class II Catechol Bioisosteres were prepared according to Scheme 3 (FIG. 4).

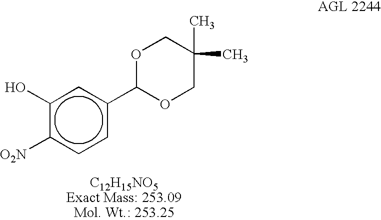

2.66 gr, 16 mM, 3-hydroxy 4-nitro benzaldehyde, 2.7 gr, 26 mM, 1,3-dihydroxy 2,2-dimethyl propane and 0.1 gr TsOH in 30 ml benzene were refluxed for 7 hours with Dean-stark azeotropic separation. Workup and recrystalization with hexane gave 2.9 gr, 72% yield, light-yellow solid, mp-58°. NMR CDCl₃ δ 10.57 (1H,s,OH), 8.10 (1H,d,J=8.2 Hz,H₅), 7.32 (1H,d,J=2.2 Hz,H₂), 7.13 (1H,dd, J=8.2, 2.2 Hz,H₆), 5.37 (1H, s, acetal), 3.70 (4H,ABq,J_{AB}=11.7 Hz), 1.26 (3H,s,methyl), 0.83(3H,s,methyl). MS m/e-(CI)-254(M⁺+1,100%), 235(M-water, 33), 223(M-NO or 2 methyl,30), 115(30). AGL 2252 (2406)

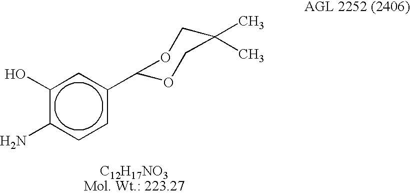

1.5 gr AG 2244 was hydrogenated with 10% Pd/C in ethanol for 4 hours. Filtering and evaporating gave 1.16 gr, 88% yield, red solid, mp-285d°. NMR CDCl₃ δ 6.89(1H,d, J=2.2 Hz,H₂), 6.85(1H,dd, J=8.0, 2.2 Hz,H₆), 6.66(1H,d, J=8.0 Hz,H₅), 5.17(1H,s, acetal), 3.68(4H,AB q,J_{AB}=11.0 Hz), 1.30(3H,s,methyl), 0.78(3H,s,methyl). MS m/e (CI)-224(M⁺+1,100%), 138(28), 115(53).

AGL 24.06—RA—NI

To 1.5 gr, 6 mM, AGL 2244, and 1 ml hydrazine hydrate in 30 ml ethanol and 10 ml water was added 100 mg Ra—Ni suspension. The reaction was refluxed for 40 minutes, decanted and worked up to give 0.7 gr, 52% yield, light-brown solid. NMR CDCl₃ δ 7.23(1H,d,J=2.2 Hz,H₂), 6.80(1H,dd,J=8.0, 2.2 Hz,H₆), 6.59 (1H,d,J=8.0 Hz,H₅), 5.17(1H,s,acetal), 3.61(4H,AB q,J_{AB}=11.0 Hz), 1.22 (3H,s,methyl), 0.73(3H,s,methyl).

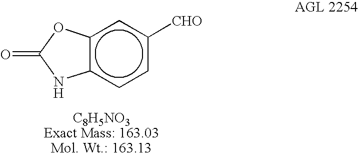

To 0.67 gr, 3 mM, AGL 2252, and 0.3 gr NaHCO₃ in 25 ml water and 25 ml ethanol was added 0.5 gr ClCOOPh. After 20 minutes 0.15 gr NaOH in 20 ml water was added. After 0.5 hours HCl was added until acidic pH and the reaction worked up to give after trituration with hexane 213 mg, mp-191°. NMR acetone d₆ δ 9.97(1H,s,CHO), 7.81(1H,dd,J=8.0, 2.2 Hz,H₆), 7.72(1H,d,J=2.2 Hz,H₂), 7.34(1H,d,J=8.0 Hz,H₅).

AGL 2407

To 0.7 gr, 3.4 mM, AGL 2406 (from RA—Ni reduction), and 0.4 gr NaHCO₃ in 25 ml water and 25 ml ethanol was added 0.6 gr, 3.8 mM, ClCOOPh. After 30 minutes at room temperature 0.3 gr NaOH in 15 ml water was added (note double amount of NaOH). After 0.5 hours HCl was added until acidic pH and the reaction worked up to give after trituration with hexane 335 mg, 78% yield. NMR-insoluble in chloroform. NMR CDCl₃ δ lightly soluble 9.95(1H,s,CHO), 7.74(1H,dd,J=8.0, 2.2 Hz,H₆), 7.22(1H,d,J=2.2 Hz,H₂), 6.84(1H,d,J=8.0 Hz,H₅). NMR acetone-identical to AGL 2254.

The following Class II Para Isomer catechol bioisostere compounds were prepared: 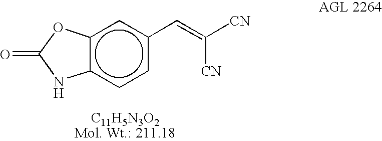
R═CN

70 mg, 0.5 mM, AGL 2254, 51 mg, 0.8 mM malononitrile and 10 mg β-alanine in 20 ml ethanol were refluxed for 4 hours. Evaporation and trituration with dichloromethane gave 80 mg, 74% yield, yellow-solid, mp-182°. NMR acetone d₆ δ 8.29(1H,s,vinyl), 7.95(1H,d,J=1.8 Hz,H₂), 7.89(1H,dd,J=8.4, 1.8 Hz,H₆), 7.40(1H, d, J=8.4 Hz,H₅).

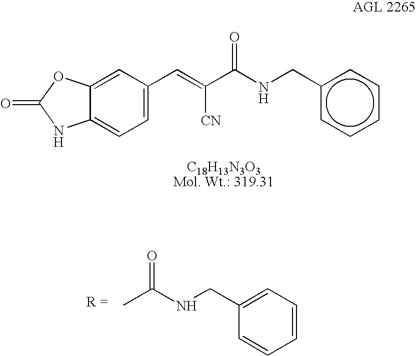

70 mg, 0.5 mM, AGL 2253, 80 mg, 0.46 mM, AG 477 and 10 mg β-alanine in 20 ml ethanol were refluxed for 3 hours. Evaporation and trituration with dichloromethane gave 82 mg, 50% yield, light brown solid, mp-258°. NMR acetone d₆ δ 8.28(1H,s,vinyl), 7.98(1H,d,J=2.2 Hz,H₂), 7.85 (1H,dd,J=8.0, 2.2 Hz,H₆), 7.46 (6H,m), 4.60(2H,s).

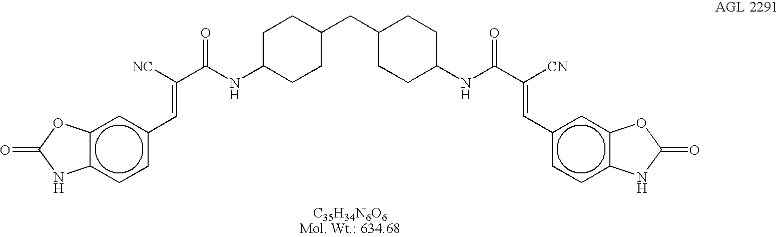

50 mg, 0.3 mM, AGL 2254, 50 mg, 0.15 mM AG 585 and 10 mg β-alanine in 10 ml ethanol were refluxed for 3 hours. Cooling and filtering gave 48 mg, 50% yield, light-yellow solid, mp-282°. NMR DMSO d₆ δ 8.06(2H,s,vinyl), 7.82(2H,d,J=2.0 Hz,H₂), 7-0.69 (2H,dd,J=8.0, 2.0 Hz,H₆), 7.18(2H,d,J=8.0 Hz,H₅), 1.83-1.0(22H,m).

The following compounds can be prepared using similar methods:

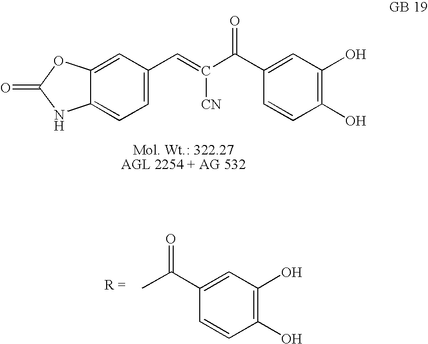

### Example 4

Preparation of Catechol Bioisosteres—Class II-Meta Isomers

Meta Isomers of Class II Catechol Bioisosteres were prepared according to Scheme 4 (FIG. 5).

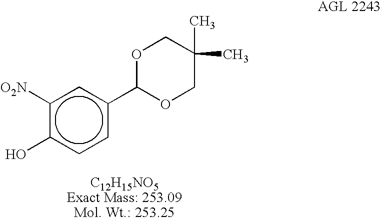

1.64 gr, 10 mM 3-nitro 4-hydroxy benzaldehyde, 1.5 gr, 14 mM 1,3-dihydroxy 2,2-dimethyl propane and 0.1 gr TsOH in 30 ml benzene were refluxed for 16 hours with Dean-stark azeotropic separation. Workup and recrystallization with hexane gave 1.55 gr, 61% yield, light-yellow solid, mp-45°. NMR CDCl₃ δ 10.63 (1H,s,OH), 8.27(1H,d,J=2.2 Hz,H₂), 7.77(1H,dd,J=8.8, 2.2 Hz,H₆), 7.16 (1H,d,J=8.8 Hz,H₅), 5.37(1H,s,acetal), 3.70(4H,AB q,J_{AB}=11.0 Hz), 1.29(3H,s,methyl), 0.83(3H,s,methyl). MSm/e(CI)-253(M⁺,25%), 223(M-NO, 11), 201(100%), 186(22), 177(14), 132(15).

3.3 gr aldehyde, 3.3 gr diol and 0.2 gr TsOH, 6 hours gave 4.2 gr, 86% yield. 10.6 gr aldehyde, 10 gr diol and 1 gr TsOh, 12 hours gave 12.1 gr, 75% yield. 3-nitro 4-hydroxybenzaldehyde NMR CDCl₃ δ 10.56 (1H,s,OH), 9.95 (1H,S, CHO), 8.64(1H,d,J=2 Hz,H₂), 8.14(1H,dd,J=8.2 Hz,H₆), 7.34(1H,d,J=8.8 Hz,H₅).

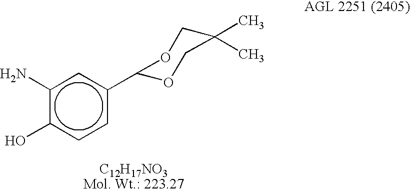

1.47 gr AG 2243 was hydrogenated with 10% Pd/C in ethanol for 6 hours. Filtering and evaporating gave 0.82 gr, 55% yield, light-green solid, mp-125°. (hydrogenation with atmospheric pressure failed). NMR CDCl₃ δ 6.94 (1H,d, J=2.2 Hz,H₂), 6.80(1H,dd,J=8.0, 2.2 Hz,H₆), 6.69(1H,d,J=8.0 Hz,H₅), 5.27(1H,s, acetal), 3.61(4H,AB q,J_{AB}=11.0 Hz), 1.28(3H,s,methyl), 0.78(3H,s,methyl). MS m/e (CI)-224(M⁺+1,100%), 138(32), 115(46).

Hydrogenation of 4.1 gr 3 hours gave 2.5 gr gray solid, 69% yield.

AGL 2405-RA—NI

To 1.5 gr, 6 mm, AGL 2243, and 1 ml hydrazine hydrate in 30 ml ethanol and 10 ml water was added 100 mg Ra—Ni suspension. The reaction was refluxed 40 minutes, decanted and worked up to give 0.75 gr, 58% yield, white solid, identical (TLC,NMR) to AGL 2251.

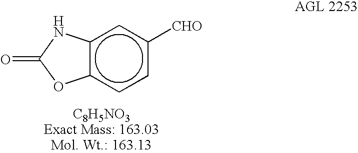

To 0.67 gr, 3 mM, AGL 2251, and 0.3 gr NaHCO₃ in 25 ml water and 25 ml ethanol was added 0.5 gr, 3.2 mM ClCOOPh. After 20 minutes 0.15 gr NaOH in 20 ml water was added. After 0.5 hours HCl was added until acidic pH and the reaction worked up to give after trituration with hexane 284 mg, 69% yield, mp-163°.NMR CDCl₃ δ 9.95(1H,s,CHO), 7.70(1H,dd,J=8.0, 2.2 Hz,H₆), 7.63(1H,d,J=2.2 Hz,H₂), 7.35(1H,d,J=8.0 Hz,H₅). NMR acetone d₆ δ 9.99(1H,s,CHO), 7.77(1H,dd,J=8.2, 1.9 Hz,H₆), 7.64(1H,d,J=1.9 Hz,H₂), 7.46(1H,d,J=8.2 Hz,H₅).

AGL 2407

To 0.75 gr, 3.4 mM, AGL 2405 (from RA—Ni reduction), and 0.4 gr NaHCO₃ in 25 ml water and 25 ml ethanol was added 0.6 gr, 3.8 mM, ClCOOPh. After 30 minutes at room temperature 0.3 gr NaOH in 15 ml water was added (note double amount of NaOH). After 0.5 hours HCl was added until acidic pH and the reaction worked up to give after trituration with hexane 335 mg, 78% yield, mp-162°. NMR CDCl₃ δ 9.96(1H,s,CHO), 8.34(br, s), 7.70(1H,dd,J=8.0, 2.2 Hz,H₆), 7.62(1H,d,J=2.2 Hz,H₂), 7.37 (1H,d,J=8.0 Hz,H₅). NMR acetone d₆ δ 9.99(1H,s,CHO), 7.76(1H,dd,J=8.2, 1.9 Hz,H₆), 7.65(1H,d,J=1.9 Hz,H₂), 7.45(1H,d,J=8.2 Hz,H₅).

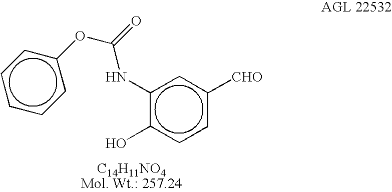

In one batch, using less NaOH gave the intermediate product, without ring closure. To 2.5 gr, 11.2 mM, AGL 2251, and 1.2 gr NaHCO₃ in 50 ml water and 50 ml ethanol was added 2 gr, 12.8 mM, ClCOOPh. After 20 minutes 0.6 gr NaOH in 20 ml water was added. After 0.5 hours HCl was added until acidic pH and the reaction worked up to give after trituration with hexane 1 gr, 65% yield. NMR acetone d₆ δ 10.02 (1H,s,CHO), 7.74 (1H,dd,J=8.2, 1.9 Hz,H₆), 7.64(1H,d,J=1.9 Hz,H₂), 7.42(1H,d,J=8.2 Hz,H₅), 7.2(3H,m), 6.80(2H,m).

The following Class II Meta Isomer catechol bioisostere compounds were prepared:

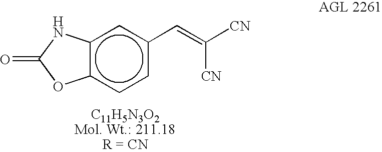

70 mg, 0.5 mM AGL 2253, 51 mg, 0.8 mM, malononitrile and 10 mg β-alanine in 20 ml ethanol were refluxed for 4 hours. Evaporation and trituration with dichloromethane gave 70 mg, 65% yield, yellow solid, mp-198° C. NMR acetone d₆ δ 8.31(1H,s,vinyl), 7.91(1H,d,J=2.2 Hz,H₂), 7.78 (1H,dd,J=8.0, 2.2 Hz,H₆), 7.46(1H,d,J=8.0 Hz,H₅).

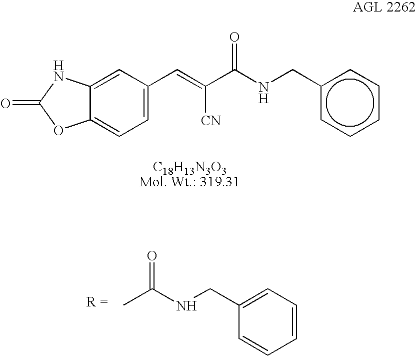

70 mg, 0.5 mM, AGL 2253, 80 mg, 0.46 mM, AG 477 and 10 mg β-alanine in 20 ml ethanol were refluxed for 4 hours. Evaporation and trituration with dichloromethane gave 84 mg, 51% yield, light-brown solid, mp-232°. NMR acetone d₆ δ 8.28(1H,s,vinyl), 7.91(1H,d,J=2.2 Hz,H₂), 7.74 (1H,dd,J=8.0, 2.2 Hz,H₆), 7.46(6H,m), 4.60(2H,s).

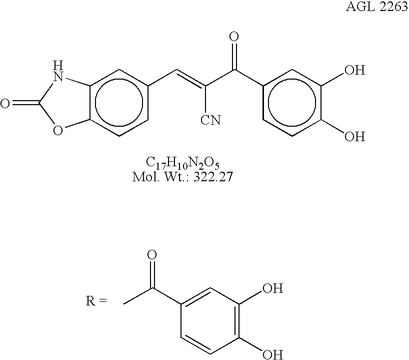

6 mg, 0.04 mM AGL 2253, 7 mg, 0.05 mM, AG 532 and 1 mg β-alanine in 20 ml ethanol were refluxed for 4 hours. Evaporation and trituration with acetone-hexane gave 2.5 mg, 15% yield, yellow solid, mp-210°. NMR acetone d₆ δ 8.06(1H,s,vinyl), 8.06(1H,d,J=1.7 Hz), 7.81(1H,dd,J=8.0, 1.7 Hz), 7.60-6.60 (4H,m).

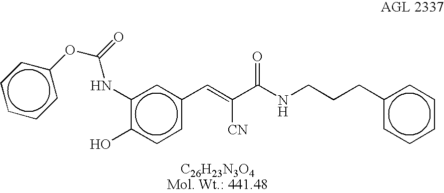

95 mg, 0.4 mM AGL 22532,145 mg, 0.7 mM, AG 552 and 10 mg β-alanine in 10 ml ethanol were refluxed 4 hours. Cooling and filtering gave 38 mg, 21% yield, light-yellow solid, mp-205°. NMR acetone d₆ δ 8.22(1H,s,vinyl), 7.89(1H,d,J=1.6 Hz,H₂), 7.72 (1H,dd,J=8.4, 1.6 Hz,H₆), 7.41(1H,d,J=8.4 Hz,H₅), 7.30(5H,m), 3.43(2H,t,J=7.0 Hz), 2.70(2H,t,J=7.0 Hz), 1.95(2H,quintet, J=7.0 Hz).

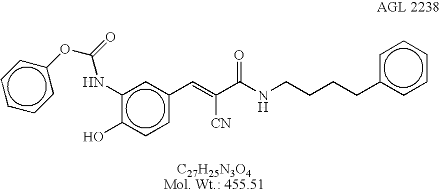

113 mg, 0.44 mM AGL 22532, 190 mg, 0.87 mM AG 553 and 10 mg β-alanine in 10 ml ethanol were refluxed for 4 hours. Cooling and filtering gave 35 mg, 24% yield, light-yellow solid, mp-174°. NMR acetone d₆ δ 8.22(1H,s,vinyl), 7.89(1H,d,J=1.6 Hz,H₂), 7.72 (1H,dd,J=8.4, 1.6 Hz,H₆), 7.41(1H,d,J=8.4 Hz,H₅), 7.30(5H,m), 3.45(2H,t,J=7.0 Hz), 2.65(2H,t,J=7.0 Hz), 1.70(2H,m).

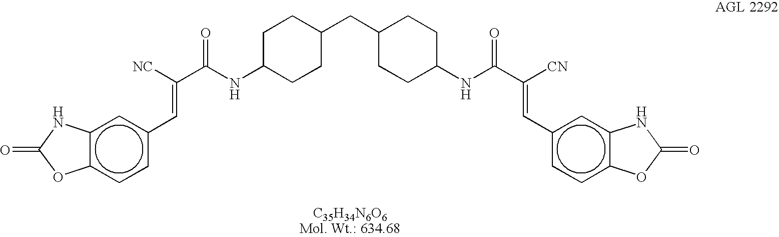

80 mg, 0.5 mM AGL 2253, 86 mg, 0.25 mM AG 585 and 10 mg β-alanine in 10 ml ethanol were refluxed 3 hours. Cooling and filtering gave 100 mg, 63% yield, light-yellow solid, mp-245°. NMR DMSO d₆ δ 8.20(2H,s,vinyl), 7.88(2H,d,J=2.0 Hz,H₂), 7.71 (2H,dd,J=8.0, 2.0 Hz,H₆), 7.41 (2H,d,J=8.0 Hz,H₅), 1.83-1.0(22H,m).

### Example 5

In Vitro Activity of Bioisosteres

The bioisosteres above were prepared to substitute the catechol rings of AG538, that is an Insulin-like Growth Factor 1 Receptor (IGF-1R) inhibitor (Blum G. et al. 2000). The in vitro activity of the compound is listed below in Table 1.

TABLE 1

| | IC50 of IGF-1R | |
|---|---|---|
| Compound | inhibition in vitro | |
| AG 538 | 60 | nM |
| AGL 2263 | 70 | nM |
| GB 16-10 | 1.2 | μM |
| GB16-11 | 200 | nM |
| GB 17 | 12 | μM |
| GB18 | 4.2 | μM |

It will be appreciated by persons skilled in the art that the present invention is not limited by what has been particularly shown and described herein above. Rather the scope of the invention is defined by the claims which follow:

1. A compound represented by the structure of formula 1: 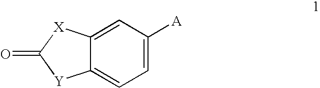
wherein X and Y are independently NR₁ or O, wherein R₁ is H or alkyl;
A is a group represented by the formula: 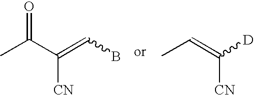
B is phenyl which is unsubstituted or substituted by one or more OR₂ or COOR₃ wherein R₂ and R₃ are independently hydrogen or alkyl, or B is a group represented by the formula: 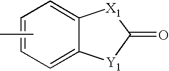
wherein X₁ and Y₁ are independently NR₁ or O, wherein R₁ is H or alkyl;
D is CN; C(═O)R₄ wherein R₄ is an alkyl, aralkyl or aryl which is unsubstituted or substituted by one or more OR₅, wherein R₅ is hydrogen or alkyl; or C(═O)NR₆R₇ wherein R₆ and R₇ are independently hydrogen or an optionally substituted alkyl, aralkyl or aryl.

2. A compound according to claim 1, represented by the structure of formula 2.

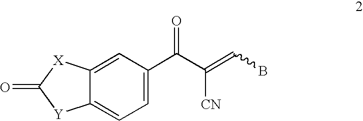

3. A compound according to claim 2, represented by the structure of formula 3. 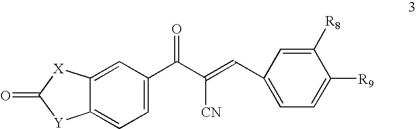
wherein R₈, R₉ are independently hydroxy, alkoxy or COOH.

4. A compound according to claim 2, represented by the structure of formula 4.

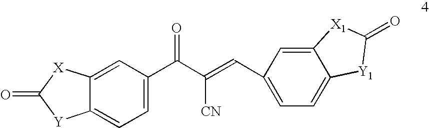

5. A compound according to claim 2, wherein said compound is selected from the group consisting of:

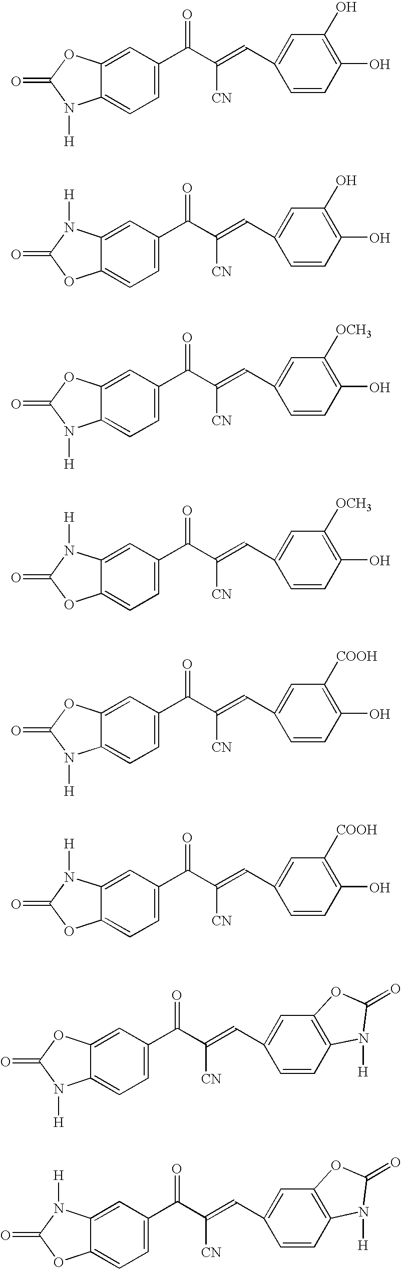

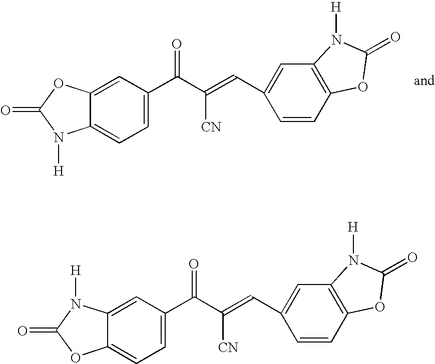

6. A compound according to claim 1, represented by the structure of formula 5.

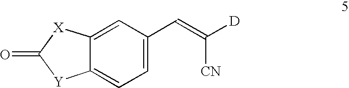

7. A compound according to claim 6, wherein said compound is selected from the group consisting of:

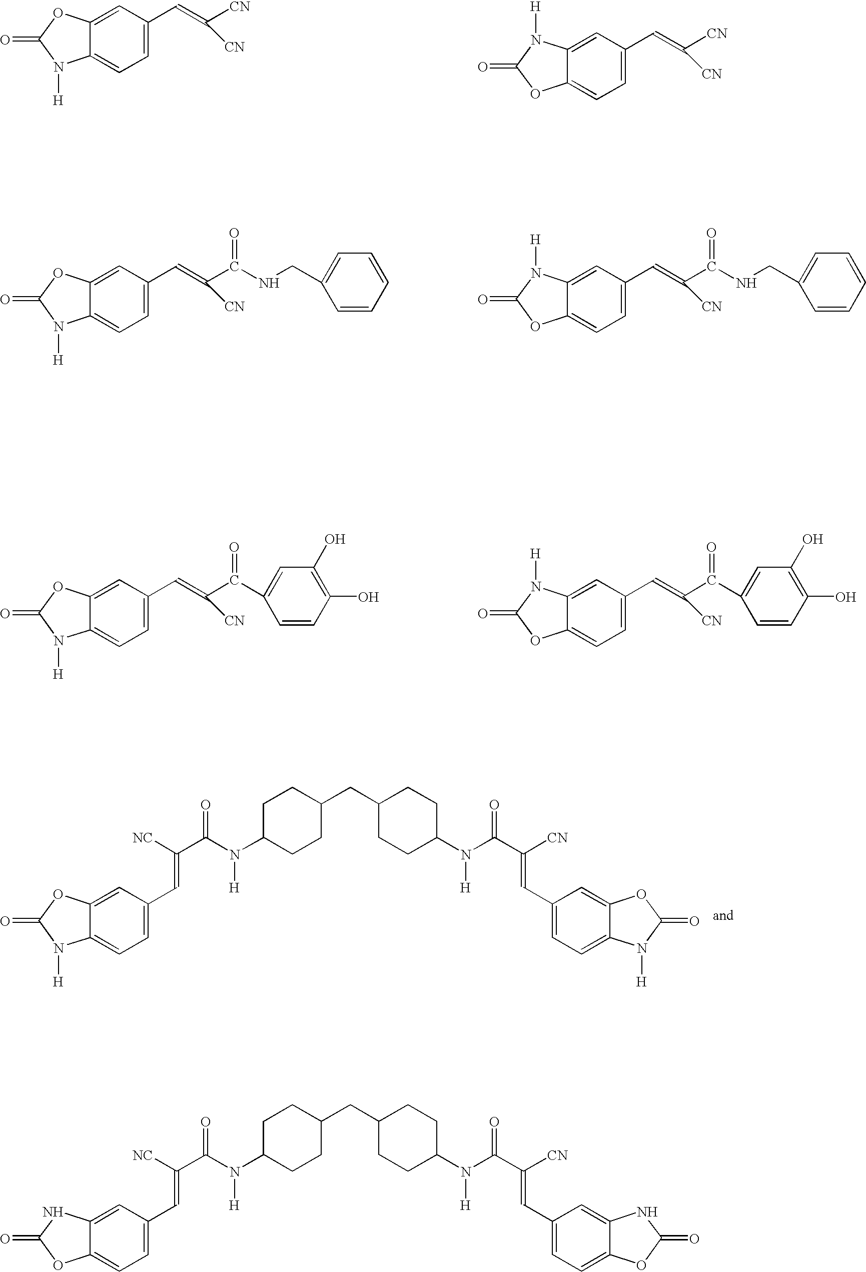

8. A compound according to claim 1, wherein X is O and Y is NR₁.

9. A compound according to claim 1, herein X is NR₁ and Y is O.

10. A compound according to claim 1, wherein X is O and Y is NH.

11. A compound according to claim 1, wherein X is NH and Y is O.

12. A compound according to claim 1, wherein X and Y are NR₁.

13. A compound according to claim 1, wherein X and Y are NH.

14. A compound according to claim 1, wherein X and Y are O.

15. A compound according to claim 1, wherein X₁ is O and Y₁ is NR₁.

16. A compound according to claim 1, wherein X₁ is NR₁ and Y₁ is O.

17. A compound according to claim 1, wherein X₁ is O and Y₁ is NH.

18. A compound according to claim 1, wherein X₁ is NH and Y₁ is O.

19. A compound according to claim 1, wherein X₁ and Y₁ are NR₁.

20. A compound according to claim 1, wherein X₁ and Y₁ are NH.

21. A compound according to claim 1, wherein X₁ and Y₁ are O.

22. A pharmaceutical composition comprising the compound according to claim 1; and a pharmaceutically acceptable carrier or excipient.

23. A method of inhibiting a protein tyrosine kinase (PTK), comprising the step of contacting said PTK with an effective inhibitory amount of a compound according to claim 1.

24. The method according to claim 23, wherein said protein tyrosine kinase is a receptor protein tyrosine kinase (RTK).

25. The method according to claim 24, wherein said receptor protein tyrosine kinase is selected from the group consisting of a platelet-derived growth factor receptor (PDGFr), a fibroblast growth factor receptor (FGF), a hepatocyte growth factor receptor (HGFr), an insulin receptor, an insulin-like growth factor-1 receptor (IGF-1r), a nerve growth factor receptor (NGF), a vascular endothelial growth factor receptor (VEGFr), and a macrophage colony stimulating factor receptor (M-CSFr).

26. The method according to claim 23, wherein said compound is selected from the group consisting of:

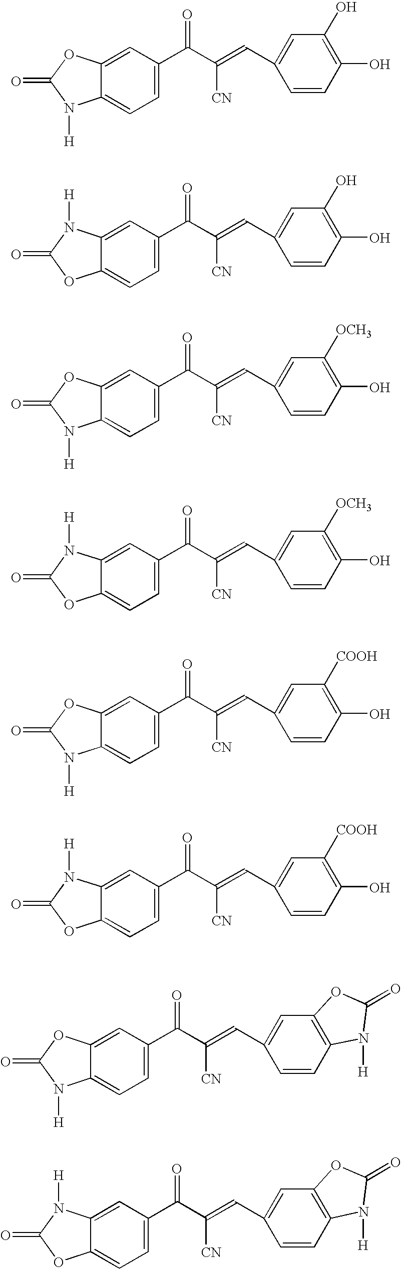

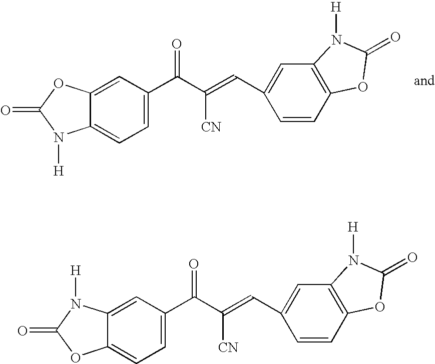

27. The method according to claim 23, wherein said compound is selected from the group consisting of:

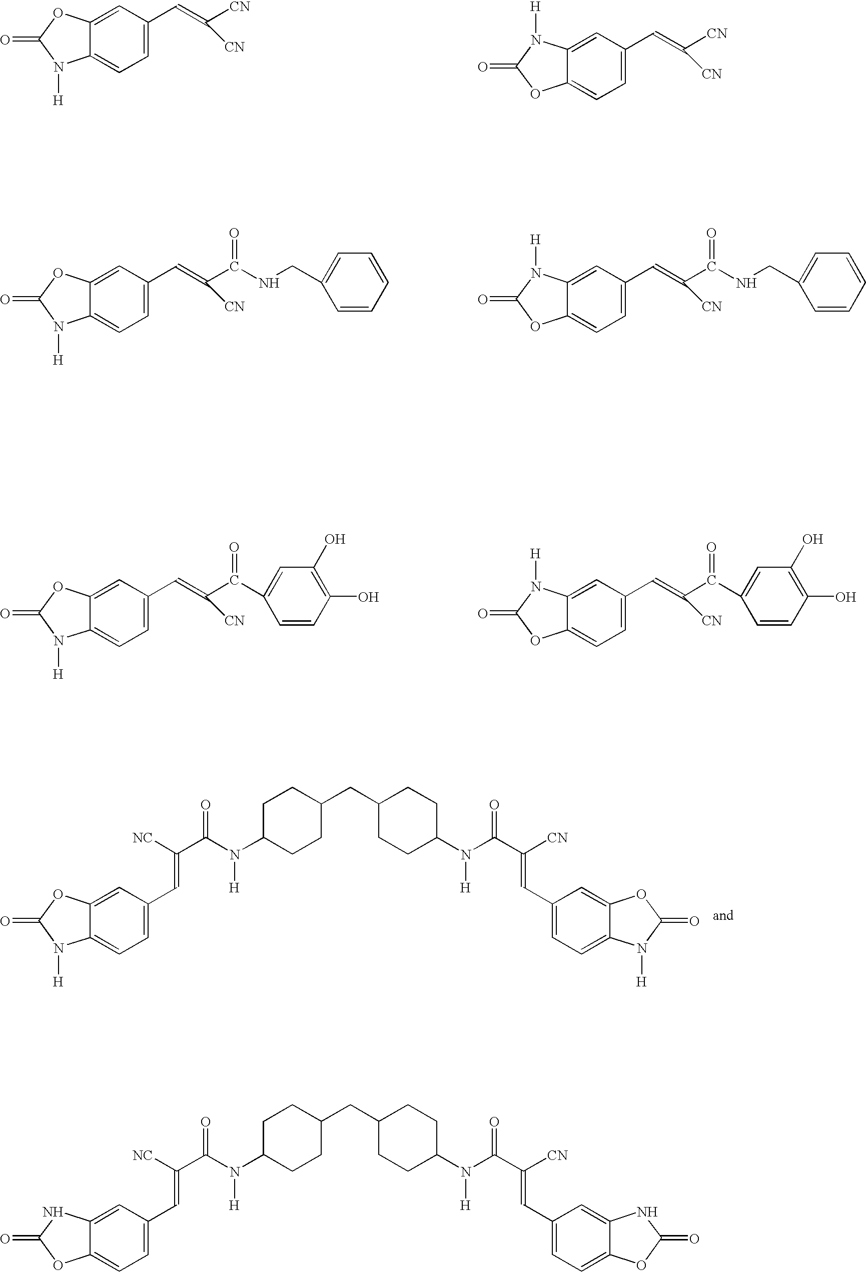

28. A method of inhibiting a protein tyrosine kinase (PTK) in a subject, comprising the step of administering to said subject a therapeutically effective amount of a compound according to claim 1.

29. The method according to claim 28, wherein said protein tyrosine kinase is a receptor protein tyrosine kinase (RTK).

30. The method according to claim 29, wherein said receptor protein tyrosine kinase is selected from the group consisting of a platelet-derived growth factor receptor (PDGFr), a fibroblast growth factor receptor (FGF), a hepatocyte growth factor receptor (HGFr), an insulin receptor, an insulin-like growth factor-1 receptor (IGF-1r), a nerve growth factor receptor (NGF), a vascular endothelial growth factor receptor (VEGFr), and a macrophage colony stimulating factor receptor (M-SFr).

31. The method according to claim 28, wherein said compound is selected from the group consisting of:

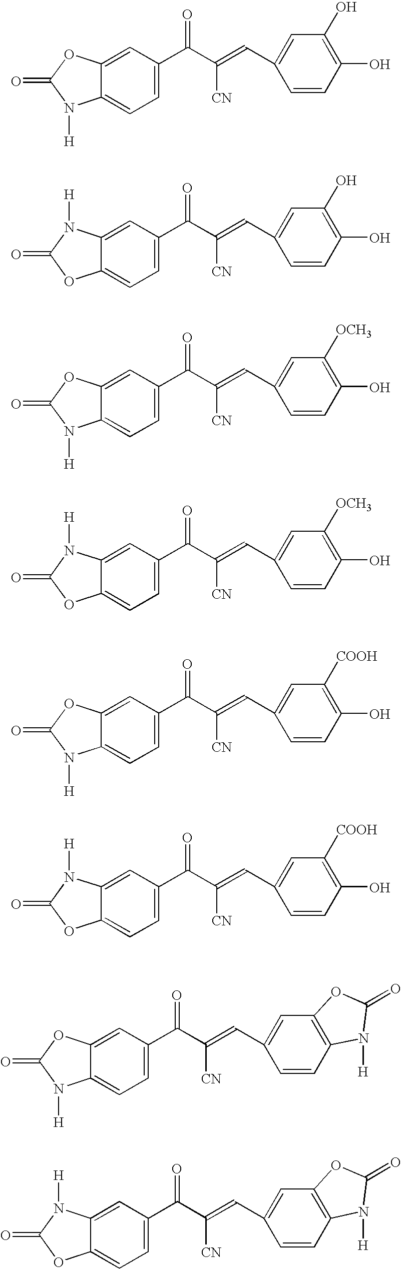

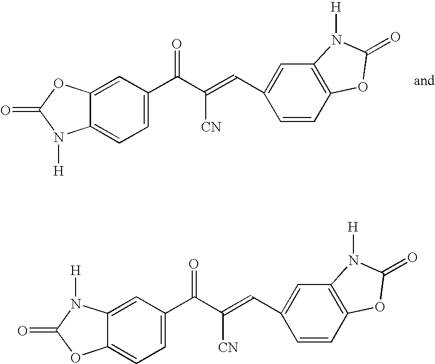

32. The method according to claim 28, wherein said compound is selected from the group consisting of:

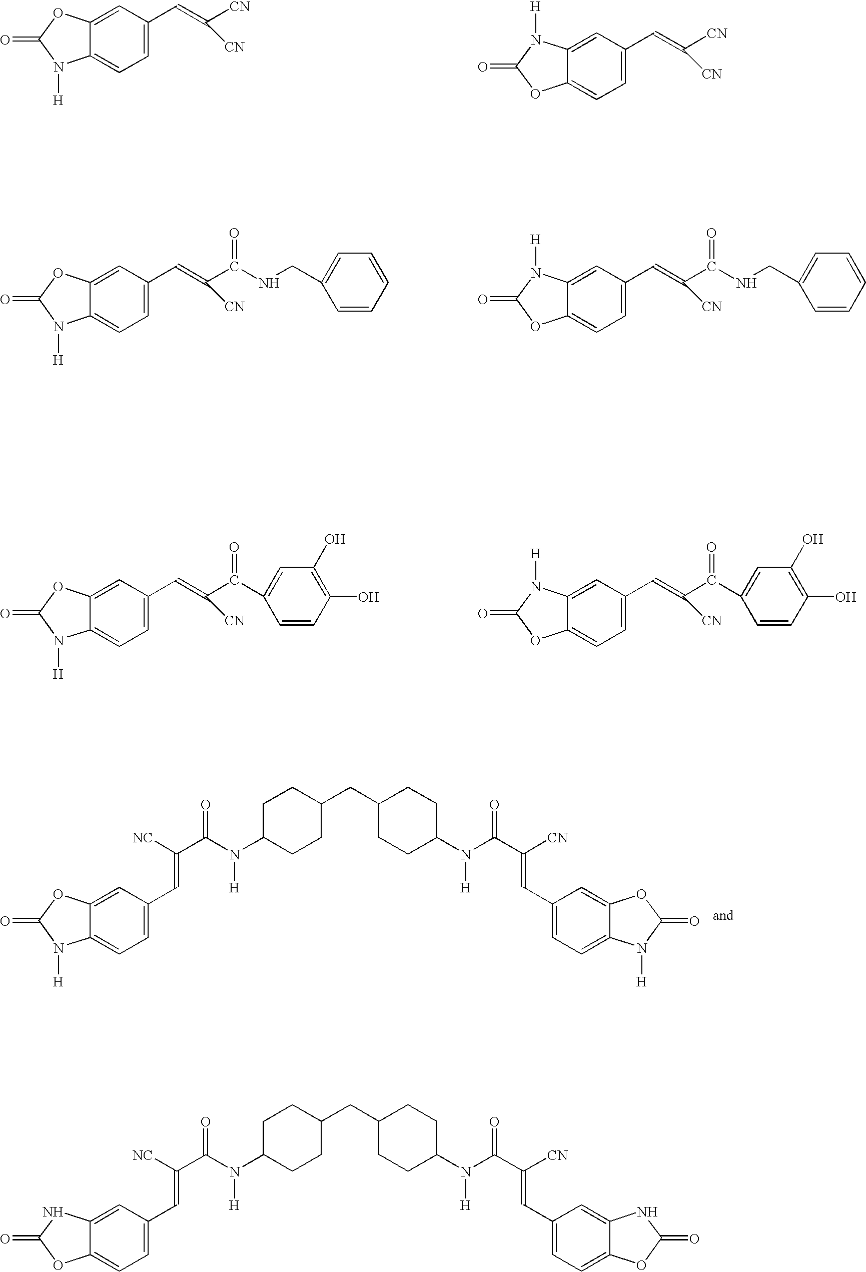

33. The method according to claim 28, comprising administering a pharmaceutical composition comprising said compound; and a pharmaceutically acceptable carrier.

34. A method of treating or preventing a protein tyrosine kinase (PTK) related disorder in a subject comprising the step of administering to said subject a therapeutically effective amount of a compound according to claim 1.

35. The method according to claim 34, wherein said protein tyrosine kinase is a receptor protein tyrosine kinase (RTK).

36. The method according to claim 35, wherein said receptor protein tyrosine kinase is selected from the group consisting of a platelet-derived growth factor receptor (PDGFr), a fibroblast growth factor receptor (FGF), a hepatocyte growth factor receptor (HGFr), an insulin receptor, an insulin-like growth factor-1 receptor (IGF-1r), a nerve growth factor receptor (NGF), a vascular endothelial growth factor receptor (VEGFr), and a macrophage colony stimulating factor receptor (M-CSFr).

37. The method according to claim 34, wherein said compound is selected from the group consisting of:

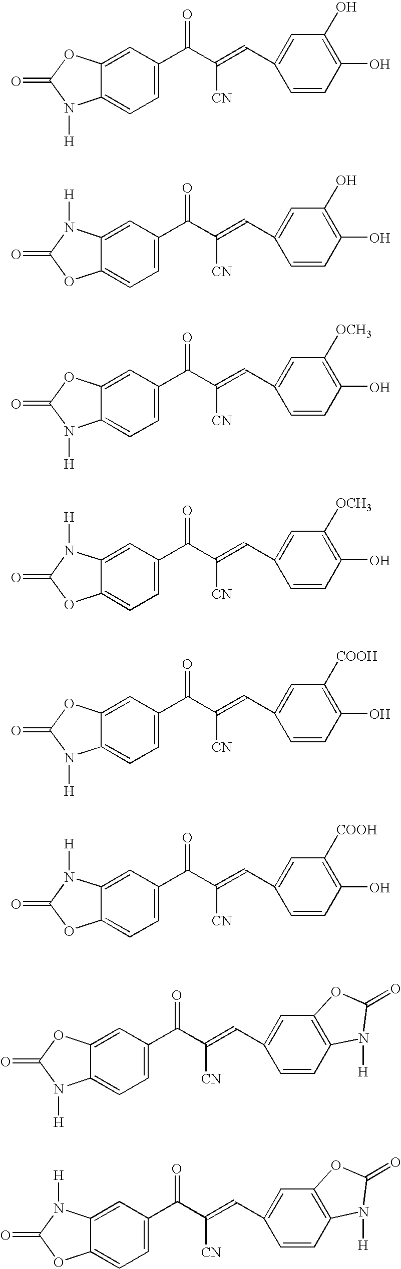

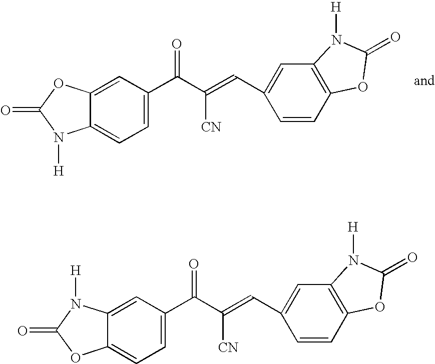

38. The method according to claim 34, wherein said compound is selected from the group consisting of:

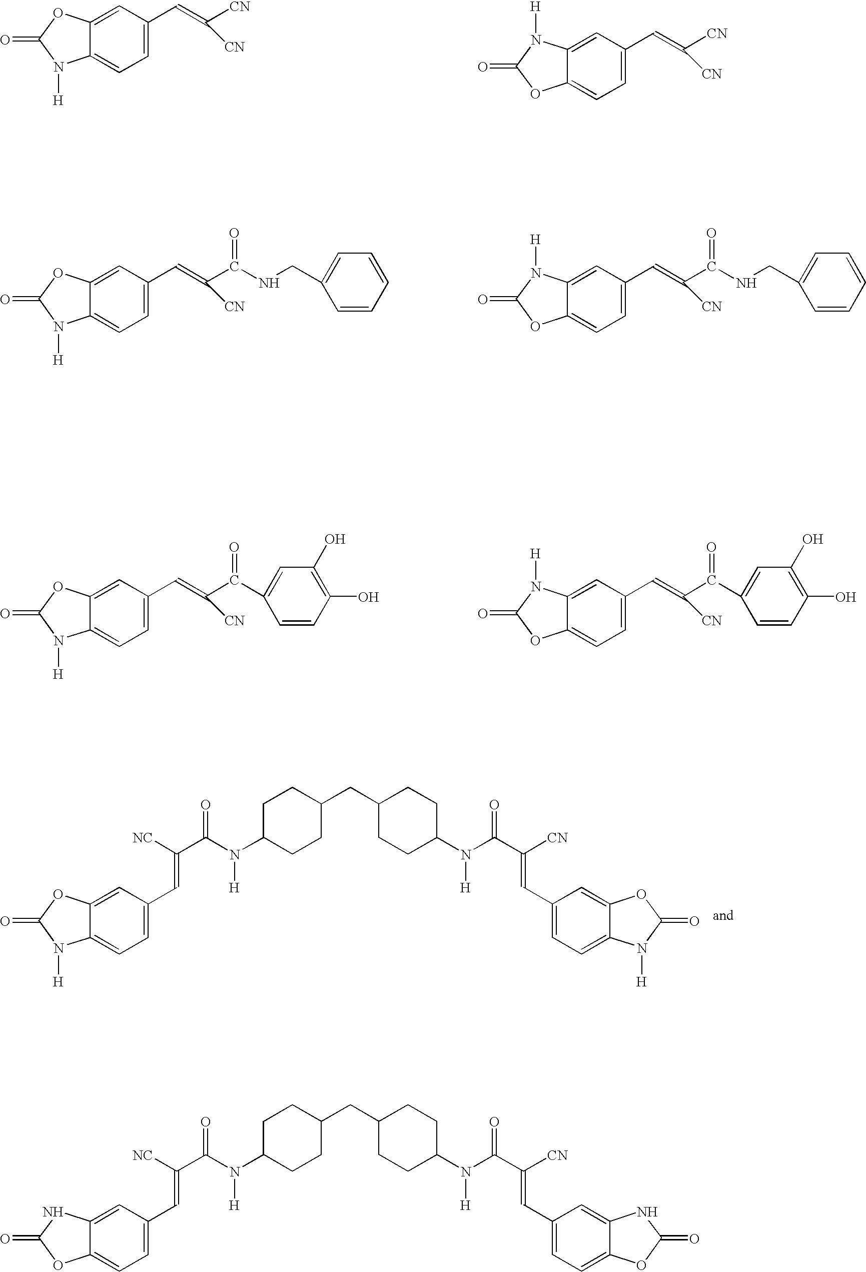

39. The method according to claim 34, wherein the PTK related disorder is a cell proliferative disorder, a fibrotic disorder, or a metabolic disorder.

40. The method according to claim 34, wherein the PTK related disorder is cancer.

41. The method according to claim 34, comprising administering a pharmaceutical composition comprising said compound; and a pharmaceutically acceptable carrier.
